(19) United States
(12) Patent Application Publication
(10) Pub. No.: US 2014/0106390 A1
(43) Pub. Date: Apr. 17, 2014

(54) BIOMARKERS FOR THE TREATMENT OF MULTIPLE MYELOMA

(71) Applicants: Mohamed Zaki, Audubon, PA (US); Christian Jacques, Hamburg, NJ (US)

(72) Inventors: Mohamed Zaki, Audubon, PA (US); Christian Jacques, Hamburg, NJ (US)

(73) Assignee: CELGENE CORPORATION, Summit, NJ (US)

(21) Appl. No.: 14/112,474

(22) PCT Filed: Apr. 17, 2012
(86) PCT No.: PCT/US12/33924

§ 371 Date: Dec. 17, 2013

Related U.S. Application Data
(60) Provisional application No. 61/476,560, filed on Apr. 18, 2011.

(51) Int. Cl. G01N 33/68

(52) U.S. Cl. CPC G01N 33/6893
USPC 435/29

(57) ABSTRACT

Provided herein are the biomarkers for predicting or monitoring the efficacy of a treatment for multiple myeloma. The use of certain M-protein or other protein levels as biomarkers to predict whether a multiple myeloma treatment is likely to be successful is also provided. Further, the analysis of these biomarkers can be used to monitor progress of treatment effectiveness and patient compliance in multiple myeloma patients who are receiving treatment.

This application claims the benefit of U.S. provisional application No. 61/476,560, filed Apr. 18, 2011, the entirety of which is incorporated herein by reference.

### 1. FIELD

Provided herein is monitoring of specific biomarkers in samples obtained from patients before and during therapy with an immunomodulatory compound alone or in combination with a second active agent for the treatment of multiple myeloma. Also provided herein is monitoring of expression of one or more specific genes, polypeptides, proteins, or antibodies during the therapy.

### 2. BACKGROUND

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin. M-protein, short for monoclonal protein, also known as paraprotein, is a particularly abnormal protein produced by the myeloma plasma cells and can be found in the blood or urine of almost all patients with multiple myeloma.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

Immunomodulatory drugs such as lenalidomide (Revlimid®) have emerged as important options for the treatment of myeloma in newly diagnosed patients, in patients with advanced disease who have failed chemotherapy or transplantation, and in patients with relapsed or refractory multiple myeloma. Another potent immunomodulatory agent is 4-(amino)-2-(2,6-dioxo(3-piperidyl))-isoindoline-1,3-dione (pomalidomide, Actimid®). In some cases, such agents are used in combination with standard chemotherapy agents. For example, lenalidomide in combination with dexamethasone was recently approved for the treatment of patients with multiple myeloma who have received at least one prior therapy. Pomalidomide may also be administered in combination with dexamethasone. Accordingly, a need exists for reliable biomarkers for multiple myeloma that can provide accurate assessment with regard to prognosis and efficacy of a particular treatment.

### 3. SUMMARY

Provided herein are biomarkers for predicting or monitoring the efficacy of a treatment for multiple myeloma. In one embodiment, provided herein is a method of predicting or monitoring the efficacy of a treatment for multiple myeloma by measuring the level of one or more specific biomarkers in samples obtained from patients before or during the treatment. In one embodiment, the samples are obtained via blood or urine. In another embodiment, the biomarkers include, but are not limited to, M-protein, albumin, creatinine, hemoglobin, beta-2-microglobulin, and combinations thereof. In one embodiment, the treatment is administration of an immunomodulatory compound provided herein elsewhere.

In yet another embodiment, a method for monitoring patient compliance with a drug treatment protocol is provided. The method comprises obtaining a biological sample from the patient, measuring the expression level of at least one biomarker provided herein in the sample, and determining if the expression level is increased or decreased in the patient sample compared to the expression level in a control untreated sample, wherein an increased or decreased expression indicates patient compliance with the drug treatment protocol.

In yet another embodiment, a kit useful for predicting the likelihood of an effective treatment of multiple myeloma is provided. Such a kit can employ, for example a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an oral tissue, gastrointestinal tissue, an organ, an organelle, a biological fluid, a blood sample, a urine sample, or a skin sample.

### 4. DETAILED DESCRIPTION

Provided herein are based, in part, on the discovery that the presence and level of certain molecules or proteins in patient samples can be utilized as biomarkers to indicate the effectiveness or progress of a treatment for multiple myeloma. In particular, these biomarkers can be used to predict, assess, and track the effectiveness of patient treatment or to monitor the patient's compliance to the treatment regimen.

### 4.1 BRIEF DESCRIPTION OF FIGURES

FIG. 1 illustrates M-protein levels in dexamethasone arms of referenced clinical studies.

FIG. 2 illustrates representative fits of individual patients (dots depict observed, light lines depict population predictions, dark lines depict individual predictions).

FIG. 3 illustrates a predictive check of the final dexamethasone tumor growth inhibition model.

FIG. 4 illustrates M-protein levels in patients treated with pomalidomide single agent in both Phase I and Phase II parts of pomalidomide study.

FIG. 5 illustrates representative fits of individual patients (dots depict observed, light lines depict population predictions, dark lines depict individual predictions).

FIG. 6 illustrates a predictive check of the final pomalidomide TGI model.

FIG. 7 illustrates survival by quartiles of week 8 M-protein change from baseline.

FIG. 8 illustrates a predictive check of the final survival model.

FIG. 9 illustrates PFS by quartiles of week 8 M-protein change from baseline.

FIG. 10 illustrates a predictive check of the final PFS models.

FIG. 11 illustrates an external evaluation of the final survival model using lenalidomide clinical data.

FIG. 12 illustrates an external evaluation of the final PFS model (“lenalidomide-dexamethasone arm” model) using lenalidomide clinical data.

FIG. 13 illustrates M-protein levels in the Phase II part of pomalidomide clinical study.

FIG. 14 illustrates predicted M-protein relative change from baseline at end of cycle 2 (week 8).

FIG. 15 illustrates simulation of expected median PFS and 95% CI for pomalidomide single agent and pomalidomide plus dexamethasone.

FIG. 16 illustrates simulation of expected median survival and 95% CI for pomalidomide single agent and pomalidomide plus dexamethasone.

### 4.2 Definitions

As used herein, and unless otherwise specified, the terms “treat,” “treating” and “treatment” refer to an action that occurs while a patient is suffering from multiple myeloma, which reduces the severity of myeloma, or retards or slows the progression of the cancer.

The term “sensitivity” and “sensitive” when made in reference to treatment is a relative term which refers to the degree of effectiveness of a treatment compound in lessening or decreasing the symptoms of the disease being treated. For example, the term “increased sensitivity” when used in reference to treatment of a cell or patient refers to an increase of, at least a 5%, or more, in the effectiveness in lessening or decreasing the symptoms of multiple myeloma when measured using any methods well-accepted in the art.

As used herein, and unless otherwise specified, the term “therapeutically effective amount” of a compound is an amount sufficient to provide a therapeutic benefit in the treatment or management of multiple myeloma, or to delay or minimize one or more symptoms associated with multiple myeloma. A therapeutically effective amount of a compound means an amount of therapeutic agent, alone or in combination with other therapies, which provides a therapeutic benefit in the treatment or management of multiple myeloma. The term “therapeutically effective amount” can encompass an amount that improves overall therapy, reduces or avoids symptoms or causes of multiple myeloma, or enhances the therapeutic efficacy of another therapeutic agent.

As used herein, an “effective patient response” refers to any increase in the therapeutic benefit to the patient such as improved survival and progression-free survival (PFS). An “effective patient tumor response” can be, for example, a 5%, 10%, 25%, 50%, or 100% decrease in the physical symptoms of multiple myeloma.

The term “likelihood” generally refers to an increase in the probability of an event. The term “likelihood” when used in reference to the effectiveness of a patient response generally contemplates an increased probability that the symptoms of multiple myeloma will be lessened or decreased.

The term “predict” generally means to determine or tell in advance. When used to “predict” the effectiveness of a multiple myeloma treatment, for example, the term “predict” can mean that the likelihood of the outcome of the treatment can be determined at the outset, before the treatment has begun, or before the treatment period has progressed substantially.

The term “monitor,” as used herein, generally refers to the overseeing, supervision, regulation, watching, tracking, or surveillance of an activity. For example, the term “monitoring the efficacy of a treatment for multiple myeloma” refers to tracking the effectiveness in treating multiple myeloma in a patient or in a cell, usually obtained from a patient. Similarly, the term “monitoring,” when used in connection with patient compliance, either individually, or in a clinical trial, refers to the tracking or confirming that the patient is actually following the treatment regimen being tested as prescribed.

As used herein the terms “polypeptide” and “protein” as used interchangeably herein, refer to a polymer of amino acids of three or more amino acids in a serial array, linked through peptide bonds. The term “polypeptide” includes proteins, protein fragments, protein analogues, oligopeptides and the like. The term polypeptide as used herein can also refer to a peptide. The amino acids making up the polypeptide may be naturally derived, or may be synthetic. The polypeptide can be purified from a biological sample.

The term “antibody” is used herein in the broadest sense and covers fully assembled antibodies, antibody fragments which retain the ability to specifically bind to the antigen (e.g., Fab, F(ab′)2, Fv, and other fragments), single chain antibodies, diabodies, antibody chimeras, hybrid antibodies, bispecific antibodies, humanized antibodies, and the like. The term “antibody” covers both polyclonal and monoclonal antibodies.

The level of a polypeptide, protein, or antibody biomarker from a patient sample can be increased as compared to a non-treated control. This increase can be about 5%, 10%, 20%, 30%, 40%, 50%, 60%, 70%, 90%, 100%, 200%, 300%, 500%, 1,000%, 5,000% or more of the comparative control protein level. Alternatively, the level of a polypeptide, protein, or antibody biomarker can be decreased. This decrease can be, for example, present at a level of about 99%, 95%, 90%, 80%, 70%, 60%, 50%, 40%, 30%, 20%, 10%, 1% or less of the comparative control protein level.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The terms “isolated” and “purified” refer to isolation of a substance (such as protein) such that the substance comprises a substantial portion of the sample in which it resides, i.e., greater than the substance is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated M-protein can typically comprise at least about 1% total M-protein. Techniques for purifying polynucleotides are well known in the art and include, for example, gel electrophoresis, ion-exchange chromatography, affinity chromatography, flow sorting, and sedimentation according to density.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood, urine, PBMCs, tissue biopsies, and the like.

4.2.1 Clinical Trial Endpoints for Cancer Approval

“Overall survival” is defined as the time from randomization until death from any cause, and is measured in the intent-to-treat population. Overall survival should be evaluated in randomized controlled studies. Demonstration of a statistically significant improvement in overall survival can be considered to be clinically significant if the toxicity profile is acceptable, and has often supported new drug approval.

Several endpoints are based on tumor assessments. These endpoints include disease free survival (DFS), objective response rate (ORR), time to progression (TTP), progression-free survival (PFS), and time-to-treatment failure (TTF). The collection and analysis of data on these time-dependent endpoints are based on indirect assessments, calculations, and estimates (e.g., tumor measurements).

Generally, “disease free survival” (DFS) is defined as the time from randomization until recurrence of tumor or death from any cause. Although overall survival is a conventional endpoint for most adjuvant settings, DFS can be an important endpoint in situations where survival may be prolonged, making a survival endpoint impractical. DFS can be a surrogate for clinical benefit or it can provide direct evidence of clinical benefit. This determination is based on the magnitude of the effect, its risk-benefit relationship, and the disease setting. The definition of DFS can be complicated, particularly when deaths are noted without prior tumor progression documentation. These events can be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

“Objective response rate” (ORR) is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study. If available, standardized criteria should be used to ascertain response. A variety of response criteria have been considered appropriate (e.g., RECIST criteria) (Therasse et al., (2000) J. Natl. Cancer Inst, 92: 205-16). The significance of ORR is assessed by its magnitude and duration, and the percentage of complete responses (no detectable evidence of tumor).

“Time to progression” (TTP) and “progression-free survival” (PFS) have served as primary endpoints for drug approval. TTP is defined as the time from randomization until objective tumor progression; TTP does not include deaths. PFS is defined as the time from randomization until objective tumor progression or death. Compared with TTP, PFS is the preferred regulatory endpoint. PFS includes deaths and thus can be a better correlate to overall survival. PFS assumes patient deaths are randomly related to tumor progression. However, in situations where the majority of deaths are unrelated to cancer, TTP can be an acceptable endpoint.

As an endpoint to support drug approval, PFS can reflect tumor growth and be assessed before the determination of a survival benefit. Its determination is not confounded by subsequent therapy. For a given sample size, the magnitude of effect on PFS can be larger than the effect on overall survival. However, the formal validation of PFS as a surrogate for survival for the many different malignancies that exist can be difficult. Data are sometimes insufficient to allow a robust evaluation of the correlation between effects on survival and PFS. Cancer trials are often small, and proven survival benefits of existing drugs are generally modest. The role of PFS as an endpoint to support licensing approval varies in different cancer settings. Whether an improvement in PFS represents a direct clinical benefit or a surrogate for clinical benefit depends on the magnitude of the effect and the risk-benefit of the new treatment compared to available therapies.

“Time-to-treatment failure” (TTF) is defined as a composite endpoint measuring time from randomization to discontinuation of treatment for any reason, including disease progression, treatment toxicity, and death. TTF is not recommended as a regulatory endpoint for drug approval. TTF does not adequately distinguish efficacy from these additional variables. A regulatory endpoint should clearly distinguish the efficacy of the drug from toxicity, patient or physician withdrawal, or patient intolerance.

### 4.3 BIOMARKERS

Provided herein are methods relating to the use of proteins, and cell marker molecules as biomarkers to predict or ascertain the efficacy of a treatment for multiple myeloma. M-protein or other protein levels can be used to determine whether a treatment is likely to be successful in models of disease.

A biological marker or “biomarker” is a substance whose detection indicates a particular biological state, such as, for example, the progress of multiple myeloma. In some embodiments, biomarkers can either be determined individually, or several biomarkers can be measured simultaneously.

4.3.1 Use of Proteins as Biomarkers for Predicting Efficacy

Based, in part, on the finding that detectable increase or decrease in certain proteins are observed during multiple myeloma treatment, the levels of these proteins may be used as a biomarker for predicting the sensitivity of a potential multiple myeloma treatment. The proteins, immunoglobulins, or antibodies include, but are not limited to: M-protein, albumin, creatinine, hemoglobin, and beta-2-microglobulin. Each of these biomarkers may be monitored separately, or two or more of the biomarkers may be monitored simultaneously.

In some embodiments, these biomarkers can be used to predict the effectiveness of a multiple myeloma treatment in a patient. In one embodiment, the level of the biomarker is measured in a biological sample obtained from a potential patient. Alternatively, the cell markers can also be used as a biomarker for an in vitro assay to predict the success of a multiple myeloma treatment, by taking a sample of cells from the patient, culturing them in the presence or absence of the treatment compound, and testing the cells for an increase or decrease in the levels of the biomarkers.

Thus, in one embodiment, provided herein is a method of monitoring tumor response to treatment in a multiple myeloma patient, comprising:

obtaining a biological sample from the patient;

measuring the level of biomarker selected from the group consisting of M-protein, albumin, creatinine, hemoglobin, beta-2-microglobulin, and combinations thereof in the biological sample;

administering an immunomodulatory compound to the patient;

thereafter obtaining a second biological sample from the patient;

measuring the level of biomarker in the second biological sample; and

comparing the levels biomarker;

wherein a decreased level of biomarker after treatment indicates the likelihood of an effective tumor response.

In one embodiment, the treatment compound is an immunomodulatory compound provided herein elsewhere. In another embodiment, the treatment compound is 4-amino-2-(2,6-dioxopiperidin-3-yl)isoindole-1,3-dione. In other embodiments, the treatment further comprises administration of dexamethasone.

In another embodiment, provided herein is a method of predicting tumor response to treatment in a multiple myeloma patient, comprising:

obtaining tumor cells from the patient;

culturing the cells in the presence or absence of an immunomodulatory compound;

measuring the level of biomarker selected from the group consisting of M-protein, albumin, creatinine, hemoglobin, beta-2-microglobulin, and combinations thereof in the tumor cells; and

comparing the levels of said biomarker in tumor cells cultured in the presence of an immunomodulatory compound to levels of biomarker in tumor cells cultured in the absence of an immunomodulatory compound;

wherein a decreased level of biomarker in the presence of an immunomodulatory compound indicates the likelihood of an effective patient tumor response to the immunomodulatory compound.

4.3.2 Use of Proteins as Biomarkers for Monitoring Efficacy or Patient Compliance

In addition to the initial prediction of the likelihood of treatment effectiveness in a patient with multiple myeloma, the progress of a multiple myeloma treatment can be followed by monitoring the levels of the proteins, immunoglobulins, or antibodies described above. Thus, in some embodiments, a method of assessing or monitoring the effectiveness of a multiple myeloma treatment in a patient is provided. A sample is obtained from the patient, and the levels of one or more of the above-described biomarkers are measured to determine whether their levels are increased or decreased compared to the levels prior to the initiation of the treatment.

The biomarkers can also be used to track and adjust individual patient treatment effectiveness. The biomarkers can be used to gather information needed to make adjustments in a patient's treatment, increasing or decreasing the dose of an agent as needed. For example, a patient receiving a treatment compound can be tested using a biomarker to see if the dosage is becoming effective, or if a more aggressive treatment plan may be needed.

In another embodiment, provided herein is a method for monitoring patient compliance with a drug treatment protocol for multiple myeloma, comprising:

obtaining a biological sample from said patient;

measuring the level of biomarker selected from the group consisting of M-protein, albumin, creatinine, hemoglobin, beta-2-microglobulin, and combinations thereof in said sample; and

determining if the level of biomarker is decreased in the patient sample compared to the level of the biomarker in an untreated control sample;

wherein a decreased level indicates patient compliance with said drug treatment protocol.

### 4.4 IMMUNOMODULATORY COMPOUNDS

In some embodiments, the biomarkers provided herein may be used to predict or monitor the efficacy of treatment for multiple myeloma by an immunomodulatory compound. The immunomodulatory compounds, including compounds known as “IMiDs®” (Celgene Corporation), are a group of compounds that can be useful to treat several types of human diseases, including certain cancers. As provided herein, these compounds can be effective in treating multiple myeloma. In some embodiments, an immunomodulatory compound can be administered to a cell sample or to a patient, and the effectiveness of the treatment can be followed using M-protein or other protein biomarkers as described herein.

As used herein and unless otherwise indicated, the term “immunomodulatory compound” can encompass certain small organic molecules that inhibit LPS induced monocyte TNF-α, IL-1β, IL-12, IL-6, MIP-1α, MCP-1, GM-CSF, G-CSF, and COX-2 production. These compounds can be prepared synthetically, or can be obtained commercially.

Exemplary immunomodulating compounds include but are not limited to N-{[2-(2,6-dioxo(3-piperidyl)-1,3-dioxoisoindolin-4-yl]methyl}cyclopropyl-carboxamide; 3-[2-(2,6-dioxo-piperidin-3-yl)-1,3-dioxo-2,3-dihydro-1H-isoindol-4-ylmethyl]-1,1-dimethyl-urea; (−)-3-(3,4-Dimethoxy-phenyl)-3-(1-oxo-1,3-dihydro-isoindol-2-yl)-propionamide; (+)-3-(3,4-Dimethoxy-phenyl)-3-(1-oxo-1,3-dihydro-isoindol-2-yl)-propionamide; (−)-{2-[1-(3-ethoxy-4-methoxyphenyl)-2-methylsulfonylethyl]-4-acetylaminoisoindoline-1,3-dione}; (+)-{2-[1-(3-ethoxy-4-methoxyphenyl)-2-methylsulfonylethyl]-4-acetylaminoisoindoline-1,3-dione}; Difluoro-methoxy SelCIDs; 1-phthalimido-1-(3,4-diethoxyphenyl)ethane; 3-(3,4-dimethoxyphenyl)-3-(3,5-dimethoxyphenyl)acrylo nitrile; 1-oxo-2-(2,6-dioxopiperidin-3-yl)-4-aminoisoindoline; 1,3-dioxo-2-(2,6-dioxopiperidin-3-yl)-4-aminoisoindoline; 4-amino-2-(3-methyl-2,6-dioxo-piperidine-3-yl)-isoindole-1,3-dione; 3-(3-acetoamidophthalimido)-3-(3-ethoxy-4-methoxyphenyl)-N-hydroxypropionamide; 1-oxo-2-(2,6-dioxopiperidin-3-yl)-4-methylisoindoline; Cyclopropyl-N-{2-[(1S)-1-(3-ethoxy-4-methoxyphenyl)-2-(methylsulfonyl)ethyl]-3-oxoisoindoline-4-yl}carboxamide; Substituted 2-(3-hydroxy-2,6-dioxopiperidin-5-yl)isoindoline; N-[2-(2,6-Dioxo-piperidin-3-yl)-1,3-dioxo-2,3-dihydro-1H-isoindol-5-ylmethyl]-4-trifluoromethoxybenzamide; (S)-4-chloro-N-((2-(3-methyl-2,6-dioxopiperidin-3-yl)-1,3-dioxoisoindolin-5-yl)methyl)benzamide; Pyridine-2-carboxylic acid [2-[(3S)-3-methyl-2,6-dioxo-piperidin-3-yl]-1,3-dioxo-2,3-dihydro-1H-isoindol-5-ylmethyl]-amide; (S)—N-((2-(3-methyl-2,6-dioxopiperidin-3-yl)-1,3-dioxoisoindolin-5-yl)methyl)-4-(trifluoromethyl)benzamide; 3-(2,5-dimethyl-4-oxo-4H-quinazolin-3-yl)-piperidine-2,6-dione, and the like.

The inflammatory cytokine TNF-α, which is produced by macrophages and monocytes during acute inflammation, causes a diverse range of signaling events within cells. Without being limited by a particular theory, one of the biological effects exerted by the immunomodulatory compounds disclosed herein is the reduction of myeloid cell TNF-α production. Immunomodulatory compounds disclosed herein may enhance the degradation of TNF-α mRNA.

Further, without being limited by theory, immunomodulatory compounds disclosed herein may also be potent co-stimulators of T cells and increase cell proliferation dramatically in a dose dependent manner. Immunomodulatory compounds disclosed herein may also have a greater co-stimulatory effect on the CD8+ T cell subset than on the CD4+ T cell subset. In addition, the compounds may have anti-inflammatory properties against myeloid cell responses, yet efficiently co-stimulate T cells to produce greater amounts of IL-2, IFN-γ, and to enhance T cell proliferation and CD8+ T cell cytotoxic activity. Further, without being limited by a particular theory, immunomodulatory compounds disclosed herein may be capable of acting both indirectly through cytokine activation and directly on Natural Killer (“NK”) cells and Natural Killer T (“NKT”) cells, and increase the NK cells' ability to produce beneficial cytokines such as, but not limited to, IFN-γ, and to enhance NK and NKT cell cytotoxic activity.

Specific examples of immunomodulatory compounds include cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperidin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), substituted 2-(2,6-dioxopiperidin-3-yl) phthalimides and substituted 2-(2,6-dioxopiperidin-3-yl)-1-oxoisoindoles including, but not limited to, those disclosed in U.S. Pat. Nos. 5,635,517, 6,281,230, 6,316,471, 6,403,613, 6,476,052 and 6,555,554; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds such as those described in U.S. patent publication no. 2003/0045552 published on Mar. 6, 2003, U.S. patent publication no. 2003/0096841 published on May 22, 2003, and International Application No. PCT/US01/50401 (International Publication No. WO 02/059106). US patent publication no. 2006/0205787 describes 4-amino-2-(3-methyl-2,6-dioxopiperidin-3-yl)-isoindole-1,3-dione compositions. US patent publication no. 2007/0049618 describes isoindole-imide compounds. The entireties of each of the patents and patent applications identified herein are incorporated by reference. In one embodiment, immunomodulatory compounds do not include thalidomide.

Various immunomodulatory compounds disclosed herein contain one or more chiral centers, and can exist as racemic mixtures of enantiomers or mixtures of diastereomers. Thus, also provided herein is the use of stereomerically pure forms of such compounds, as well as the use of mixtures of those forms. For example, mixtures comprising equal or unequal amounts of the enantiomers of a particular immunomodulatory compounds may be used. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, 1N, 1972).

Immunomodulatory compounds provided herein include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference.

These compounds have the structure I:

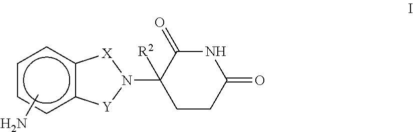

in which one of X and Y is C═O, the other of X and Y is C═O or CH₂, and R² is hydrogen or lower alkyl, in particular methyl. Specific immunomodulatory compounds include, but are not limited to:

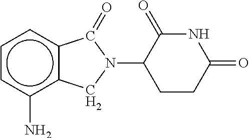

1-oxo-2-(2,6-dioxopiperidin-3-yl)-4-aminoisoindoline;

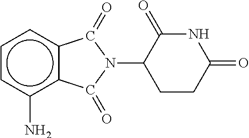

1,3-dioxo-2-(2,6-dioxopiperidin-3-yl)-4-aminoisoindoline; and

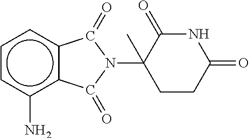

1,3-dioxo-2-(3-methyl-2,6-dioxopiperidin-3-yl)-4-aminoisoindole, and optically pure isomers thereof.

The compounds can be obtained via standard, synthetic methods (see e.g., U.S. Pat. No. 5,635,517, incorporated herein by reference). The compounds are also available from Celgene Corporation, Warren, N.J.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Representative compounds are of formula:

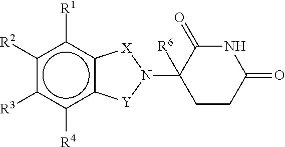

in which:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzyl, or halo;
provided that R⁶ is other than hydrogen if X and Y are C═O and (i) each of R¹, R², R³, and R⁴ is fluoro or (ii) one of R¹, R², R³, or R⁴ is amino.

Compounds representative of this class are of the formulas:

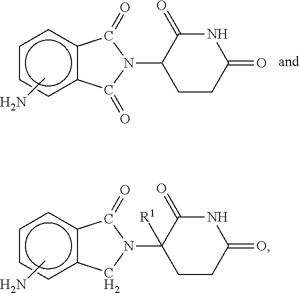

wherein R¹ is hydrogen or methyl. In a separate embodiment, provided herein is the use of enantiomerically pure forms (e.g. optically pure (R) or (S) enantiomers) of these compounds.

Still other specific immunomodulatory compounds disclosed herein belong to a class of isoindole-imides disclosed in U.S. Pat. No. 7,091,353, U.S. Patent Publication No. 2003/0045552, and International Application No. PCT/US01/50401 (International Publication No. WO 02/059106), each of which are incorporated herein by reference. Representative compounds are of formula II:

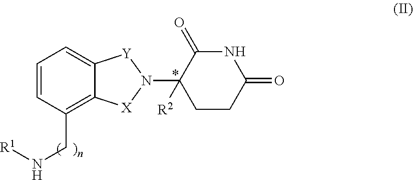

and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR^{S}, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl; each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR^{S}, C(S)NHR³, or (C₁-C₈)alkyl-O(CO)R^{5;}

R² is H or (C₁-C₈)alkyl; and
R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

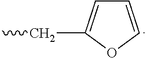

In another embodiment of the compounds of formula II, R¹ is

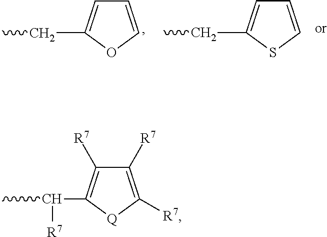

wherein Q is O or S, and each occurrence of R⁷ is independently H, (C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl]methyl}(benzylamino)carboxamide.

Still other specific immunomodulatory compounds disclosed herein belong to a class of isoindole-imides disclosed in U.S. Patent Application Publication Nos. US 2002/0045643, International Publication No. WO 98/54170, and U.S. Pat. No. 6,395,754, each of which is incorporated herein by reference. Representative compounds are of formula III:

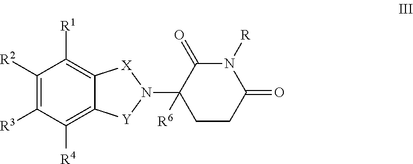

and pharmaceutically acceptable salts, hydrates, solvates, clathrates, enantiomers, diastereomers, racemates, and mixtures of stereoisomers thereof, wherein: one of X and Y is C═O and the other is CH₂ or C═O;

### R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;

### R′ is R⁷—CHR¹⁰—N(R⁸R⁹);

R⁷ is m-phenylene or p-phenylene or —(CnH2n)- in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—;
R¹⁰ is hydrogen, alkyl of to 8 carbon atoms, or phenyl; and
* represents a chiral-carbon center.

Other representative compounds are of formula:

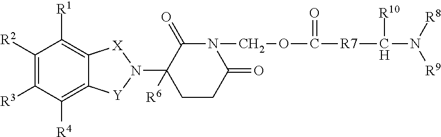

wherein:
one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CnH2n)- in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and

R¹⁰ is hydrogen, alkyl of to 8 carbon atoms, or phenyl.

Other representative compounds are of formula:

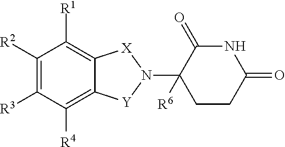

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

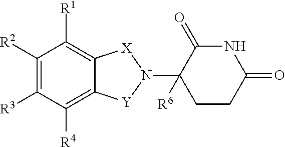

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

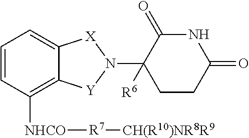

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CnH2n)- in which n has a value of 0 to 4; each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Other specific immunomodulatory compounds are 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

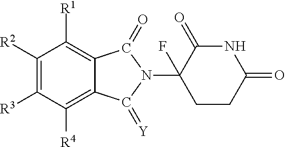

wherein:
Y is oxygen or H₂ and
each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds are the tetra substituted 2-(2,6-dioxopiperidin-3-yl)-1-oxoisoindolines described in U.S. Pat. No. 5,798,368, which is incorporated herein by reference. Representative compounds are of formula:

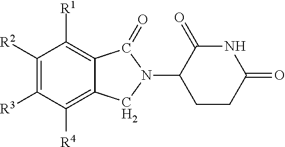

wherein each of R¹, R², R³, and R⁴, independently of the others, is halo, alkyl of 1 to 4 carbon atoms, or alkoxy of 1 to 4 carbon atoms.

Other specific immunomodulatory compounds are 1-oxo and 1,3-dioxo-2-(2,6-dioxopiperidin-3-yl) isoindolines disclosed in U.S. Pat. No. 6,403,613, which is incorporated herein by reference. Representative compounds are of formula:

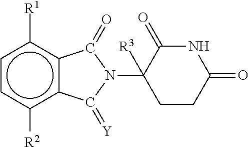

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

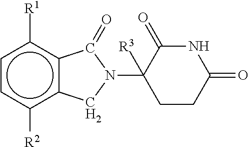

wherein
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

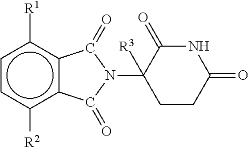

wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl.

Other specific immunomodulatory compounds disclosed herein are 1-oxo and 1,3-dioxoisoindolines substituted in the 4- or 5-position of the indoline ring described in U.S. Pat. No. 6,380,239 and U.S. Pat. No. 7,244,759, both of which are incorporated herein by reference. Representative compounds are of formula:

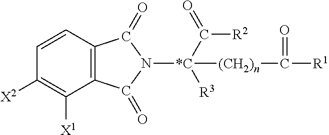

in which the carbon atom designated C* constitutes a center of chirality (when n is not zero and R¹ is not the same as R²); one of X¹ and X² is amino, nitro, alkyl of one to six carbons, or NH—Z, and the other of X¹ or X² is hydrogen; each of R¹ and R² independent of the other, is hydroxy or NH—Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

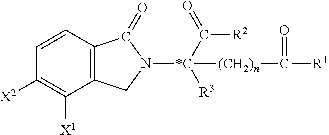

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

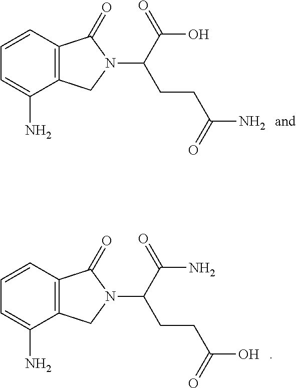 [SMILES: NC(=O)CCC(C(=O)O)N1Cc2c(N)cccc2C1=O ; NC(=O)C(CCC(=O)O)N1Cc2c(N)cccc2C1=O]

Other representative compounds are of formula:

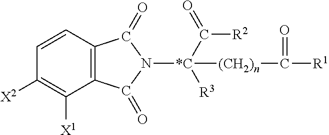

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

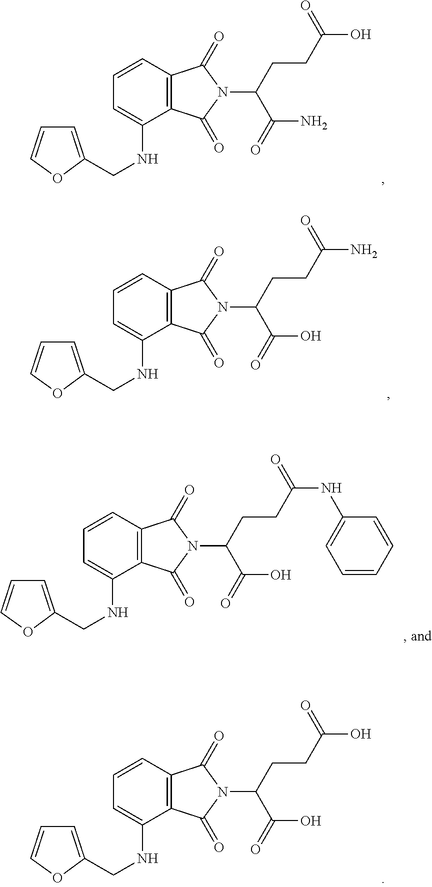 [SMILES: NC(=O)C(CCC(=O)O)N1C(=O)c2cccc(NCc3ccco3)c2C1=O ; NC(=O)CCC(C(=O)O)N1C(=O)c2cccc(NCc3ccco3)c2C1=O ; O=C(CCC(C(=O)O)N1C(=O)c2cccc(NCc3ccco3)c2C1=O)Nc1ccccc1 ; O=C(O)CCC(C(=O)O)N1C(=O)c2cccc(NCc3ccco3)c2C1=O]

Other specific examples of the compounds are of formula:

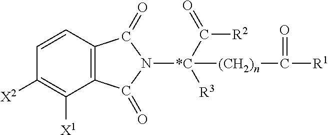

wherein:

one of X¹ and X² is nitro, or NH—Z, and the other of X¹ or X² is hydrogen;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Other representative compounds are of formula:

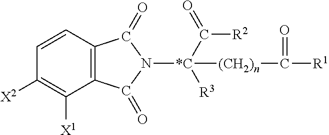

wherein:

one of X¹ and X² is alkyl of one to six carbons;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds are isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

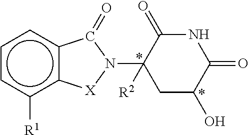

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

Further representative compounds are of formula:

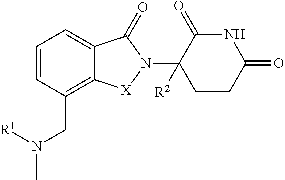

and pharmaceutically acceptable salts, solvate, stereoisomers, and prodrugs thereof, wherein:

### X is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H or (C₁-C₈)alkyl;
R³ and R^{3′} are independently (C₁-C₈)alkyl; (C₃-C₇)cycloalkyl; (C₂-C₈)alkenyl; (C₂-C₈)alkynyl; benzyl; (C₀-C₄)alkyl-(C₅-C₁₀)aryl, optionally substituted with one or more (C₁-C₆)alkyl, itself optionally substituted with one or more halogen, (C₁-C₆)alkoxy, (C₁-C₆)alkylenedioxy or halogen; (C₀-C₄)alkyl-(C₁-C₆)heterocycloalkyl; (C₀-C₄)alkyl-(C₂-C₅)heteroaryl; (C₀-C₈)alkyl-N(R⁶)₂; (C₁-C₈)alkyl-OR⁵; (C₁-C₈)alkyl-C(O)OR⁵; (C₁-C₈)alkyl-O(CO)R⁵; or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl; each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group.

In one embodiment, X is C═O. In another embodiment, X is CH₂.

In one embodiment, R¹ is H. In another embodiment, R¹ is (C₁-C₈)alkyl. In another embodiment, R¹ is (C₃-C₇)cycloalkyl. In another embodiment, R¹ is (C₂-C₈)alkenyl. In another embodiment, R¹ is (C₂-C₈)alkynyl. In another embodiment, R¹ is benzyl. In another embodiment, R¹ is aryl. In another embodiment, R¹ is (C₀-C₄)alkyl-(C₁-C₆)heterocycloalkyl. In another embodiment, R¹ is (C₀-C₄)alkyl-(C₂-C₅)heteroaryl. In another embodiment, R¹ is C(O)R³. In another embodiment, R¹ is C(S)R³. In another embodiment, R¹ is C(O)OR⁴. In another embodiment, R¹ is (C₁-C₈)alkyl-N(R⁶)₂. In another embodiment, R¹ is (C₁-C₈)alkyl-OR⁵. In another embodiment, R¹ is (C₁-C₈)alkyl-C(O)OR⁵. In another embodiment, R¹ is C(O)NHR³. In another embodiment, R¹ is C(S)NHR³. In another embodiment, R¹ is C(O)NR³R^{3′}. In another embodiment, R¹ is C(S)NR³R^{3′}. In another embodiment, R¹ is (C₁-C₈)alkyl-O(CO)R⁵.

In one embodiment, R² is H. In another embodiment, R² is (C₁-C₈)alkyl.

In one embodiment, R³ is (C₁-C₈)alkyl. In another embodiment, R³ is (C₃-C₇)cycloalkyl. In another embodiment, R³ is (C₂-C₈)alkenyl. In another embodiment, R³ is (C₂-C₈)alkynyl. In another embodiment, R³ is benzyl. In another embodiment, R³ is (C₀-C₄)alkyl-(C₅-C₁₀)aryl, optionally substituted with one or more (C₁-C₆)alkyl, itself optionally substituted with one or more halogen, (C₁-C₆)alkoxy, (C₁-C₆)alkylenedioxy or halogen. In another embodiment, R³ is (C₀-C₄)alkyl-(C₁-C₆)heterocycloalkyl. In another embodiment, R³ is (C₀-C₄)alkyl-(C₂-C₅)heteroaryl. In another embodiment, R³ is (C₀-C₈)alkyl-N(R⁶)₂. In another embodiment, R³ is (C₁-C₈)alkyl-OR⁵. In another embodiment, R³ is (C₁-C₈)alkyl-C(O)OR⁵. In another embodiment, R³ is (C₁-C₈)alkyl-O(CO)R⁵. In another embodiment, R³ is C(O)OR⁵. In one embodiment, R^{3′} is (C₁-C₈)alkyl. In another embodiment, R^{3′} is (C₃-C₇)cycloalkyl. In another embodiment, R^{3′} is (C₂-C₈)alkenyl. In another embodiment, R^{3′} is (C₂-C₈)alkynyl. In another embodiment, R^{3′} is benzyl. In another embodiment, R^{3′} is aryl. In another embodiment, R^{3′} is (C₀-C₄)alkyl-(C₁-C₆)heterocycloalkyl. In another embodiment, R^{3′} is (C₀-C₄)alkyl-(C₂-C₅)heteroaryl. In another embodiment, R^{3′} is (C₀-C₈)alkyl-N(R⁶)₂. In another embodiment, R^{3′} is (C₁-C₈)alkyl-OR⁵. In another embodiment, R^{3′} is (C₁-C₈)alkyl-C(O)OR⁵. In another embodiment, R^{3′} is (C₁-C₈)alkyl-O(CO)R⁵. In another embodiment, R^{3′} is C(O)OR⁵.

In one embodiment, R⁴ is (C₁-C₈)alkyl. In another embodiment, R⁴ is (C₂-C₈)alkenyl. In another embodiment, R⁴ is (C₂-C₈)alkynyl. In another embodiment, R⁴ is (C₁-C₄)alkyl-OR⁵. In another embodiment, R⁴ is benzyl. In another embodiment, R⁴ is aryl. In another embodiment, R⁴ is (C₀-C₄)alkyl-(C₁-C₆)heterocycloalkyl. In another embodiment, R⁴ is (C₀-C₄)alkyl-(C₂-C₅)heteroaryl.

In one embodiment, R⁵ is (C₁-C₈)alkyl. In another embodiment, R⁵ is (C₂-C₈)alkenyl. In another embodiment, R⁵ is (C₂-C₈)alkynyl. In another embodiment, R⁵ is benzyl. In another embodiment, R⁵ is aryl. In another embodiment, R⁵ is (C₂-C₅)heteroaryl.

In one embodiment, R⁶ is H. In another embodiment, R⁶ is (C₁-C₈)alkyl. In another embodiment, R⁶ is (C₂-C₈)alkenyl. In another embodiment, R⁶ is (C₂-C₈)alkynyl. In another embodiment, R⁶ is benzyl. In another embodiment, R⁶ is aryl. In another embodiment, R⁶ is (C₂-C₅)heteroaryl. In another embodiment, R⁶ is or (C₀-C₈)alkyl-C(O)O—R⁵. In another embodiment, R⁶ groups join to form a heterocycloalkyl group.

In other embodiments, this invention encompasses any combination of X, R¹, R², R³, R^{3′}, R⁴, R⁵, and/or R⁶ as set forth above.

Still other representative compounds are of formula:

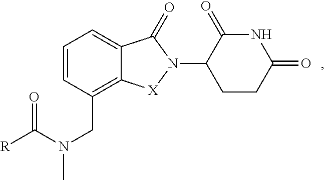

and pharmaceutically acceptable salts, solvates, stereoisomers, and prodrugs thereof, wherein:

### X is CH₂ or C═O;

R is (C₁-C₆)alkyl; (C₁-C₆)alkoxy; amino; (C₁-C₆)alkyl-amino; dialkylamino, wherein each of the alkyl groups is independently (C₁-C₆)alkyl; (C₆-C₁₀)aryl, optionally substituted with one or more (C₁-C₆)alkyl, (C₁-C₆)alkoxy or halogen; 5 to 10 membered heteroaryl, optionally substituted with one or more (C₁-C₆)alkyl; —NHR′; or (C₀-C₈)alkyl-N(R″)₂;
R′ is: (C₁-C₆)alkyl; (C₀-C₄)alkyl-(C₆-C₁₀)aryl, optionally substituted with one or more (C₁-C₆)alkyl, itself optionally substituted with one or more halogen, (C₁-C₆)alkoxy, (C₁-C₆)alkylenedioxy or halogen; or 6 to 10 membered heteroaryl, optionally substituted with one or more (C₁-C₆)alkyl; and
each occurrence of R″ is independently H, (C₁-C₈)alkyl, (C₂-C₈)alkenyl, (C₂-C₈)alkynyl, benzyl, aryl, 5 to 10 membered heteroaryl, or (C₀-C₈)alkyl-C(O)O—(C₁-C₈)alkyl.

In one embodiment, X is C═O. In another embodiment, X is CH₂.

In one embodiment, R is (C₁-C₆)alkyl. In certain specific embodiments, R is methyl, ethyl, propyl, cyclopropyl, or hexyl. In another embodiment, R is (C₁-C₆)alkoxy. In certain specific embodiments, R is t-butoxy. In another embodiment, R is amino. In another embodiment, R is (C₁-C₆)alkyl-amino. In another embodiment, R is dialkylamino, wherein each of the alkyl groups is independently (C₁-C₆)alkyl. In certain specific embodiments, R is dimethylamino. In another embodiment, R is (C₆-C₁₀)aryl, optionally substituted with one or more (C₁-C₆)alkyl, (C₁-C₆)alkoxy, or halogen. In certain specific embodiments, R is phenyl, optionally substituted with one or more methyl and/or halogen. In another embodiment, R is 5 to 10 membered heteroaryl, optionally substituted with one or more (C₁-C₆)alkyl. In certain specific embodiments, R is pyridyl or furanyl. In another embodiment, R is —NHR′.

In one embodiment, R′ is (C₁-C₆)alkyl, optionally substituted with one or more halogen. In certain specific embodiments, R′ is ethyl, propyl, t-butyl, cyclohexyl, or trifluoromethyl. In another embodiment, R′ is (C₀-C₄)alkyl-(C₆-C₁₀)aryl, optionally substituted with one or more (C₁-C₆)alkyl, (C₁-C₆)alkoxy, (C₁-C₆)alkylenedioxy or halogen. In certain specific embodiments, R′ is phenyl, optionally substituted with one or more of methyl, methoxy, and/or chloride. In another embodiment, R′ is naphthyl. In another embodiment, R′ is phenyl, substituted with (C₁-C₆)alkylenedioxy, specifically, methylenedioxy. In another embodiment, R′ is toluoyl. In another embodiment, R′ is 6 to 10 membered heteroaryl, optionally substituted with one or more (C₁-C₆)alkyl. In certain specific embodiments, R′ is pyridyl or naphthyl.

In one embodiment, R is (C₀-C₈)alkyl-N(R″)₂.

In another embodiment, R″ is H. In another embodiment, R″ is (C₁-C₈)alkyl. In another embodiment, R″ is (C₂-C₈)alkenyl. In another embodiment, R″ is (C₂-C₈)alkynyl. In another embodiment, R″ is benzyl. In another embodiment, R″ is aryl. In another embodiment, R″ is 5 to 10 membered heteroaryl. In another embodiment, R″ is (C₀-C₈)alkyl-C(O)O—(C₁-C₈)alkyl. In a specific embodiment, one of R″ is H and the other of R″ is (C₀-C₈)alkyl-C(O)O—(C₁-C₈)alkyl, in particular, —COO—isobutyl.

In other embodiments, this invention encompasses any combination of X, R, and/or R′ as set forth above.

Other representative compounds are of formula:

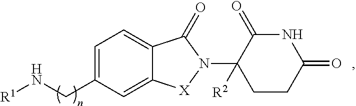

and pharmaceutically acceptable salts, solvates, stereoisomers, and prodrugs thereof, wherein:
n is 0 or 1;

### X is CH₂, C═O, or C═S;

### R¹ is:

a) —(CH₂)ₘR³ or —CO(CH₂)ₘR³, wherein
m is 0, 1, 2, or 3; and
R³ is 5-10 membered aryl or heteroaryl, optionally substituted with one or more halogen;

b) —C═YR⁴, wherein
Y is O or S; and
R⁴ is:
   (C₁-C₁₀)alkyl; (C₁-C₁₀)alkoxy;
   (C₀-C₁₀)alkyl-(5 to 10 membered heteroaryl or heterocycle), said heteroaryl or heterocycle optionally substituted with (C₁-C₆)alkyl or oxo;
   5 to 10 membered aryl, optionally substituted with one or more of: halogen;
   (C₁-C₆)alkoxy, itself optionally substituted with one or more halogen;
   or (C₁-C₆)alkyl, itself optionally substituted with one or more halogen; or
   (C₁-C₆)alkyl-CO—O—R⁵, wherein R⁵ is H or (C₁-C₆)alkyl; or

c) —C═ZNHR⁶, wherein
Z is O or S; and
R⁶ is:
   (C₁-C₁₀)alkyl; (C₁-C₁₀)alkoxy;
   5 to 10 membered aryl, optionally substituted with one or more of: halogen; cyano;
   (C1-C6)alkylenedioxo;
   (C₁-C₆)alkoxy, itself optionally substituted with one or more halogen;
   or (C₁-C₆)alkyl, itself optionally substituted with one or more halogen; and
      R² is H or (C₁-C₆)alkyl.

In one specific embodiment, this invention encompasses compounds of formula:

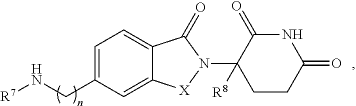

and pharmaceutically acceptable salts, solvates, stereoisomers, and prodrugs thereof, wherein:

### X is CH₂ or C═O;

R⁷ is —(CH₂)ₘR⁹ or —CO(CH₂)ₘR⁹, wherein m is 0, 1, 2, or 3, and R⁹ is 5-10 membered aryl or heteroaryl, optionally substituted with one or more halogen; and
R⁸ is H or (C₁-C₆)alkyl.

In one embodiment, X is C═O. In another embodiment, X is CH₂.

In one embodiment, R⁷ is —(CH₂)ₘR⁹. In another embodiment, R⁷ is —CO(CH₂)ₘR⁹.

In one embodiment, n is 0. In another embodiment, n is 1. In one embodiment, m is 0. In another embodiment, m is 1. In other embodiments, m is 2 or 3.

In one embodiment, R⁹ is 5-10 membered aryl. In certain specific embodiments, R⁹ is phenyl, optionally substituted with one or more halogen. In one embodiment, R⁹ is 5-10 membered heteroaryl. In certain specific embodiments, R⁹ is furyl or benzofuryl.

In one embodiment, R⁸ is H. In another embodiment, R⁸ is (C₁-C₆)alkyl. In certain specific embodiments, R⁸ is methyl.

All of the combinations of the above embodiments are encompassed by this invention.

In another embodiment, this invention encompasses compounds of formula:

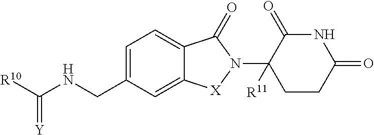

and pharmaceutically acceptable salts, solvates, stereoisomers, and prodrugs thereof, wherein:

### X is CH₂ or C═O;

### Y is O or S;

### R¹⁰ is:

(C₁-C₁₀)alkyl; (C₁-C₁₀)alkoxy;
(C₀-C₁₀)alkyl-(5 to 10 membered heteroaryl or heterocycle), said heteroaryl or heterocycle optionally substituted with (C₁-C₆)alkyl or oxo;
5 to 10 membered aryl, optionally substituted with one or more of:
   halogen; (C₁-C₆)alkoxy, itself optionally substituted with one or more halogen; or (C₁-C₆)alkyl, itself optionally substituted with one or more halogen; or
(C1-C6)alkyl-CO—O—R¹², wherein R¹² is H or (C₁-C₆)alkyl; and
   R¹¹ is H or (C₁-C₆)alkyl.

In one embodiment, X is CH₂. In another embodiment, X is C═O. In one embodiment, Y is O. In another embodiment, Y is S.

In one embodiment, R¹⁰ is (C₁-C₁₀)alkyl. In certain specific embodiments, R¹⁰ is (C₅-C₁₀)alkyl. In certain specific embodiments, R¹⁰ is pentyl or hexyl. In one embodiment, R¹⁰ is (C₁-C₁₀)alkoxy. In certain specific embodiments, R¹⁰ is (C₅-C₁₀)alkoxy. In certain specific embodiments, R¹⁰ is pentyloxy or hexyloxy. In one embodiment, R¹⁰ is 5 to 10 membered heteroaryl. In certain specific embodiments, R¹⁰ is thiopheneyl or furyl. In one embodiment, R¹⁰ is 5 to 10 membered aryl, optionally substituted with one or more halogen. In certain specific embodiments, R¹⁰ is phenyl, optionally substituted with one or more halogen. In one embodiment, R¹⁰ is 5 to 10 membered aryl, optionally substituted with (C₁-C₆)alkyl or (C₁-C₆)alkoxy, themselves optionally substituted with one or more halogen. In certain specific embodiments, R¹⁰ is phenyl substituted with (C₁-C₃)alkyl or (C₁-C₃)alkoxy, substituted with one or more halogen. In certain specific embodiments, R¹⁰ is phenyl substituted with methyl or methoxy, substituted with 1, 2, or 3 halogens. In one embodiment, R¹⁰ is (C₁-C₆)alkyl-CO—O—R¹², and R¹² is (C₁-C₆)alkyl. In one specific embodiment, R¹⁰ is butyl-CO—O-tBu. In one embodiment, R¹⁰ is (C₁-C₆)alkyl-CO—O—R¹², and R¹² is H. In one specific embodiment, R¹⁰ is butyl-COOH.

In one embodiment, R¹¹ is H. In another embodiment, R¹¹ is (C₁-C₆)alkyl. In certain specific embodiments, R¹¹ is methyl.

All of the combinations of the above embodiments are encompassed by this invention.

In another embodiment, this invention encompasses compounds of formula:

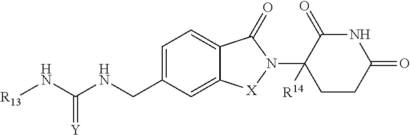

and pharmaceutically acceptable salts, solvates, stereoisomers, and prodrugs thereof, wherein:

### X is CH₂ or C═O;

### Y is O or S;

### R¹³ is:

(C₁-C₁₀)alkyl; (C₁-C₁₀)alkoxy;

5 to 10 membered aryl, optionally substituted with one or more of:
halogen; cyano; (C₁-C₆)alkylenedioxy; (C₁-C₆)alkoxy, itself optionally
substituted with one or more halogen; or (C₁-C₆)alkyl, itself optionally
substituted with one or more halogen; and
   R¹⁴ is H or (C₁-C₆)alkyl.

In one embodiment, X is CH₂. In another embodiment, X is C═O. In one embodiment, Y is O. In another embodiment, Y is S. In one embodiment, R¹³ is (C₁-C₁₀)alkyl. In certain specific embodiments, R¹³ is (C₁-C₆)alkyl. In certain specific embodiments, R¹³ is propyl, butyl, pentyl, or hexyl. In one embodiment, R¹³ is (C₁-C₁₀)alkoxy. In one embodiment, R¹³ is 5 to 10 membered aryl, optionally substituted with cyano. In certain specific embodiments, R¹³ is phenyl, optionally substituted with cyano. In one embodiment, R¹³ is 5 to 10 membered aryl, optionally substituted with (C₁-C₆)alkylenedioxy. In certain specific embodiments, R¹³ is phenyl, optionally substituted with methylenedioxy. In one embodiment, R¹³ is 5 to 10 membered aryl, optionally substituted with one or more halogen. In certain specific embodiments, R¹³ is phenyl, optionally substituted with one or more halogen. In another embodiment, R¹³ is 5 to 10 membered aryl, optionally substituted with (C₁-C₆)alkyl or (C₁-C₆)alkoxy, themselves optionally substituted with one or more halogens. In certain specific embodiments, R¹³ is phenyl, optionally substituted with methyl or methoxy, themselves optionally substituted with 1, 2, or 3 halogens.

In another embodiment, R¹⁴ is H. In another embodiment, R¹⁴ is (C₁-C₆)alkyl. In certain specific embodiments, R¹⁴ is methyl.

All of the combinations of the above embodiments are encompassed by this invention.

Representative compounds also have the formula:

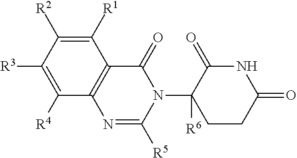

and pharmaceutically acceptable salts, solvates, and stereoisomers thereof, wherein:
R¹ is hydrogen;
each of R², R³, and R⁴ is independently: hydrogen; halo; —(CH₂)ₙOH; (C₁-C₆)alkyl, optionally substituted with one or more halo; (C₁-C₆)alkoxy, optionally substituted with one or more halo; or

—(CH₂)ₙNHR^{a}, wherein R^{a} is:
hydrogen;
(C₁-C₆)alkyl, optionally substituted with one or more halo;
—(CH₂)ₙ-(6 to 10 membered aryl);
—C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃;

(C₁-C₆)alkyl, said alkyl itself optionally substituted with one or more halo; or (C₁-C₆)alkoxy, said alkoxy itself optionally substituted with one or more halo;

—C(O)—(C₁-C₈)alkyl, wherein the alkyl is optionally substituted with one or more halo;

—C(O)—(CH₂)ₙ—(C₃-C₁₀-cycloalkyl);

—C(O)—(CH₂)ₙ—NR^{b}R^{c}, wherein R^{b} and R^{c} are each independently:
hydrogen;
(C₁-C₆)alkyl, optionally substituted with one or more halo;
(C₁-C₆)alkoxy, optionally substituted with one or more halo; or
6 to 10 membered aryl, optionally substituted with one or more of: halo;

(C₁-C₆)alkyl, itself optionally substituted with one or more halo; or

(C₁-C₆)alkoxy, itself optionally substituted with one or more halo;

—C(O)—(CH₂)ₙ—O—(C₁-C₆)alkyl; or

—C(O)—(CH₂)ₙ—O—(CH₂)ₙ-(6 to 10 membered aryl); or two of R¹-R⁴ together can form a 5 or 6 membered ring, optionally substituted with one or more of: halo; (C₁-C₆)alkyl, optionally substituted with one or more halo; and (C₁-C₆)alkoxy, optionally substituted with one or more halo;

R⁵ is: hydrogen; —(CH₂)ₙOH; phenyl; —O—(C₁-C₆)alkyl; or (C₁-C₆)alkyl, optionally substituted with one or more halo;
R⁶ is: hydrogen; or (C₁-C₆)alkyl, optionally substituted with one or more halo; and
n is 0, 1, or 2.

Further representative compounds have the formula:

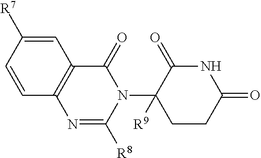

and pharmaceutically acceptable salts, solvates, and stereoisomers thereof, wherein: R⁷ is: hydrogen; halo; —(CH₂)ₙOH; (C₁-C₆)alkyl, optionally substituted with one or more halo; (C₁-C₆)alkoxy, optionally substituted with one or more halo; or

—(CH₂)ₙNHR^{d}, wherein R^{d} is:
hydrogen;
(C₁-C₆)alkyl, optionally substituted with one or more halo;
—(CH₂)ₙ-(6 to 10 membered aryl);
—C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃;
(C₁-C₆)alkyl, itself optionally substituted with one or more halo;
or (C₁-C₆)alkoxy, itself optionally substituted with one or more halo;
—C(O)—(C₁-C₈)alkyl, wherein the alkyl is optionally substituted with one or more of: halo;
—C(O)—(CH₂)ₙ—(C₃-C₁₀-cycloalkyl);
—C(O)—(CH₂)ₙ—NR^{e}R^{f}, wherein R^{e} and R^{f} are each independently: hydrogen;
(C₁-C₆)alkyl, optionally substituted with one or more halo;
(C₁-C₆)alkoxy, optionally substituted with one or more halo; or
6 to 10 membered aryl, optionally substituted with one or more of: halo; (C₁-C₆)alkyl, itself optionally substituted with one or more halo; or (C₁-C₆)alkoxy, itself optionally substituted with one or more halo;
—C(O)—(CH₂)ₙ—O—(C₁-C₆)alkyl; or
—C(O)—(CH₂)ₙ—O—(CH₂)ₙ-(6 to 10 membered aryl);
   R⁸ is: hydrogen; —(CH₂)ₙOH; phenyl; —O—(C₁-C₆)alkyl; or (C₁-C₆)alkyl, optionally substituted with one or more halo;
   R⁹ is: hydrogen; or (C₁-C₆)alkyl, optionally substituted with one or more halo; and
   n is 0, 1, or 2.

Still further representative compounds have the formula:

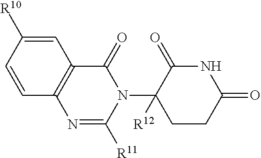

and pharmaceutically acceptable salts, solvates, and stereoisomers thereof, wherein:
R¹⁰ is: hydrogen; halo; —(CH₂)ₙOH; (C₁-C₆)alkyl, optionally substituted with one or more halo; or (C₁-C₆)alkoxy, optionally substituted with one or more halo;
R¹¹ is: hydrogen; —(CH₂)ₙOH; phenyl; —O—(C₁-C₆)alkyl; or (C₁-C₆)alkyl, optionally substituted with one or more halo;
R¹² is: hydrogen; or (C₁-C₆)alkyl, optionally substituted with one or more halo; and
n is 0, 1, or 2.

In one embodiment, R¹⁰ is hydrogen. In another embodiment, R¹⁰ is halo. In another embodiment, R¹⁰ is (C₁-C₆)alkyl, optionally substituted with one or more halo. In another embodiment, R¹⁰ is —(CH₂)ₙOH or hydroxyl. In another embodiment, R¹⁰ is (C₁-C₆)alkoxy, optionally substituted with one or more halo.

In one embodiment, R¹¹ is hydrogen. In another embodiment, R¹¹ is —(CH₂)ₙOH or hydroxyl. In another embodiment, R¹¹ is phenyl. In another embodiment, R¹¹ is —O—(C₁-C₆)alkyl, optionally substituted with one or more halo. In another embodiment, R¹¹ is (C₁-C₆)alkyl, optionally substituted with one or more halo.

In one embodiment, R¹² is hydrogen. In another embodiment, R¹² is (C₁-C₆)alkyl, optionally substituted with one or more halo.

In one embodiment, n is 0. In another embodiment, n is 1. In another embodiment, n is 2. Compounds provided herein encompass any of the combinations of R¹⁰, R¹¹, R¹² and n described above. In one specific embodiment, R¹⁰ is halo. In another embodiment, R¹⁰ is hydroxyl. In another embodiment, R¹⁰ is methyl. In another specific embodiment, R¹¹ is hydrogen. In another embodiment, R¹¹ is methyl. In another specific embodiment, R¹² is hydrogen. In another embodiment, R¹² is methyl.

In another embodiment, provided herein are compounds of formula:

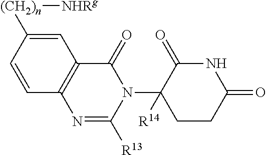

and pharmaceutically acceptable salts, solvates, and stereoisomers thereof, wherein:

### R^{g} is:

hydrogen;

(C₁-C₆)alkyl, optionally substituted with one or more halo;

—(CH₂)ₙ-(6 to 10 membered aryl);

—C(O)—(CH₂)ₙ(6 to 10 membered aryl) or —C(O)—(CH₂)ₙ(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;

—C(O)—(C₁-C₈)alkyl, wherein the alkyl is optionally substituted with one or more halo;

—C(O)—(CH₂)ₙ—(C₃-C₁₀-cycloalkyl);

—C(O)—(CH₂)ₙ—NR^{h}Rⁱ, wherein R^{h} and Rⁱ are each independently:
hydrogen;
(C₁-C₆)alkyl, optionally substituted with one or more halo;
(C₁-C₆)alkoxy, optionally substituted with one or more halo; or
6 to 10 membered aryl, optionally substituted with one or more of: halo;
(C₁-C₆)alkyl, itself optionally substituted with one or more halo; or
(C₁-C₆)alkoxy, itself optionally substituted with one or more halo;

—C(O)—(CH₂)ₙ—O—(C₁-C₆)alkyl; or

C(O)—(CH₂)ₙ—O—(CH₂)ₙ-(6 to 10 membered aryl);

R¹³ is: hydrogen; —(CH₂)ₙOH; phenyl; —O—(C₁-C₆)alkyl; or (C₁-C₆)alkyl, optionally substituted with one or more halo;
R¹⁴ is: hydrogen; or (C₁-C₆)alkyl, optionally substituted with one or more halo; and
n is 0, 1, or 2.

In one embodiment, R^{g} is hydrogen. In another embodiment, R^{g} is (C₁-C₆)alkyl, optionally substituted with one or more halo. In another embodiment, R^{g} is —(CH₂)ₙ-(6 to 10 membered aryl). In another embodiment, R^{g} is —C(O)—(CH₂)ₙ-(6 to 10 membered aryl) or —C(O)—(CH₂)ₙ-(6 to 10 membered heteroaryl), wherein the aryl or heteroaryl is optionally substituted as described above. In another embodiment, R^{g} is —C(O)—(C₁-C₈)alkyl, wherein the alkyl is optionally substituted with one or more halo. In another embodiment, R^{g} is —C(O)—(CH₂)ₙ—(C₃-C₁₀-cycloalkyl). In another embodiment, R^{g} is —C(O)—(CH₂)ₙ—NR^{h}Rⁱ, wherein R^{h} and Rⁱ are as described above. In another embodiment, R^{g} is —C(O)—(CH₂)ₙ—O—(C₁-C₆)alkyl. In another embodiment, R^{g} is —C(O)—(CH₂)ₙ—O—(CH₂)ₙ-(6 to 10 membered aryl).

In one embodiment, R¹³ is hydrogen. In another embodiment, R¹³ is —(CH₂)ₙOH or hydroxyl. In another embodiment, R¹³ is phenyl. In another embodiment, R¹³ is —O—(C₁-C₆)alkyl, optionally substituted with one or more halo. In another embodiment, R¹³ is (C₁-C₆)alkyl, optionally substituted with one or more halo.

In one embodiment, R¹⁴ is hydrogen. In another embodiment, R¹⁴ is (C₁-C₆)alkyl, optionally substituted with one or more halo.

In one embodiment, n is 0. In another embodiment, n is 1. In another embodiment, n is 2. Compounds provided herein encompass any of the combinations of R^{g}, R¹³, R¹⁴ and n described above. In one specific embodiment, R^{g} is hydrogen, and n is 0 or 1. In another embodiment, R^{g} is —C(O)—(C₁-C₆)alkyl. In another embodiment, R^{g} is —C(O)-phenyl, optionally substituted with one or more methyl, halo, and/or (C₁-C₆)alkoxy. In another specific embodiment, R¹³ is methyl. In another embodiment, R¹⁴ is hydrogen.

In another embodiment, provided herein are compounds of formula:

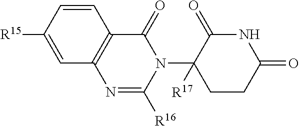

and pharmaceutically acceptable salts, solvates, and stereoisomers thereof, wherein:
R¹⁵ is: hydrogen; halo; —(CH₂)ₙOH; (C₁-C₆)alkyl, optionally substituted with one or more halo; (C₁-C₆)alkoxy, optionally substituted with one or more halo; or

—(CH₂)ₙNHR^{j}, wherein R^{j} is:
hydrogen;
(C₁-C₆)alkyl, optionally substituted with one or more halo;
—(CH₂)ₙ-(6 to 10 membered aryl);
—C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
—C(O)—(C₁-C₈)alkyl, wherein the alkyl is optionally substituted with one or more halo;
—C(O)—(CH₂)ₙ—(C₃-C₁₀-cycloalkyl); —C(O)—(CH₂)ₙ—NR^{k}R^{l}, wherein R^{k} and R^{l} are each independently:
   hydrogen;
   (C₁-C₆)alkyl, optionally substituted with one or more halo;
   (C₁-C₆)alkoxy, optionally substituted with one or more halo; or
   6 to 10 membered aryl, optionally substituted with one or more of: halo; (C₁-C₆)alkyl, itself optionally substituted with one or more halo; or (C₁-C₆)alkoxy, itself optionally substituted with one or more halo;
—C(O)—(CH₂)ₙ—O—(C₁-C₆)alkyl; or
—C(O)—(CH₂)ₙ—O—(CH₂)ₙ-(6 to 10 membered aryl);
   R¹⁶ is: hydrogen; —(CH₂)ₙOH; phenyl; —O—(C₁-C₆)alkyl; or (C₁-C₆)alkyl, optionally substituted with one or more halo;
   R¹⁷ is: hydrogen; or (C₁-C₆)alkyl, optionally substituted with one or more halo; and
   n is 0, 1, or 2.

In one embodiment, R¹⁵ is hydrogen. In another embodiment, R¹⁵ is halo. In another embodiment, R¹⁵ is (C₁-C₆)alkyl, optionally substituted with one or more halo. In another embodiment, R¹⁵ is —(CH₂)ₙOH or hydroxyl. In another embodiment, R¹⁵ is (C₁-C₆)alkoxy, optionally substituted with one or more halo. In one embodiment, R¹⁵ is —(CH₂)ₙNHR^{j}. In one embodiment, wherein R¹⁵ is —(CH₂)ₙNHR^{j}, R^{j} is hydrogen. In another embodiment, R^{j} is (C₁-C₆)alkyl, optionally substituted with one or more halo. In another embodiment, R^{j} is —(CH₂)ₙ-(6 to 10 membered aryl). In another embodiment, R^{j} is —C(O)—(CH₂)ₙ-(6 to 10 membered aryl) or —C(O)—(CH₂)ₙ-(6 to 10 membered heteroaryl), wherein the aryl or heteroaryl is optionally substituted as described above. In another embodiment, R^{j} is —C(O)—(C₁-C₈)alkyl, wherein the alkyl is optionally substituted with one or more halo. In another embodiment, R^{j} is —C(O)—(CH₂)ₙ—(C₃-C₁₀-cycloalkyl). In another embodiment, R^{j} is —C(O)—(CH₂)ₙ—NR^{k}R^{l}, wherein R^{k} and R^{l} are as described above. In another embodiment, R^{j} is —C(O)—(CH₂)ₙ—O—(C₁-C₆)alkyl. In another embodiment, R^{j} is —C(O)—(CH₂)ₙ—O—(CH₂)ₙ-(6 to 10 membered aryl).

In one embodiment, R¹⁶ is hydrogen. In another embodiment, R¹⁶ is —(CH₂)ₙOH or hydroxyl. In another embodiment, R¹⁶ is phenyl. In another embodiment, R¹⁶ is —O—(C₁-C₆)alkyl, optionally substituted with one or more halo. In another embodiment, R¹⁶ is (C₁-C₆)alkyl, optionally substituted with one or more halo.

In one embodiment, R¹⁷ is hydrogen. In another embodiment, R¹⁷ is (C₁-C₆)alkyl, optionally substituted with one or more halo.

In one embodiment, n is 0. In another embodiment, n is 1. In another embodiment, n is 2.

Compounds provided herein encompass any of the combinations of R¹⁵, R¹⁶, R¹⁷ and n described above. In one specific embodiment, R¹⁵ is methyl. In another embodiment, R¹⁵ is halo. In another embodiment, R¹⁵ is —CF₃. In another embodiment, R¹⁵ is —(CH₂)ₙNHR^{j}. In one specific embodiment wherein R¹⁵ is —(CH₂)ₙNHR^{j}, R^{j} is hydrogen, and n is 0 or 1. In another embodiment wherein R¹⁵ is —(CH₂)ₙNHR^{j}, R^{j} is —C(O)—(O)—(C₁-C₆)alkyl. In one specific embodiment, R¹⁶ is hydrogen. In another embodiment, R¹⁶ is methyl. In another specific embodiment, R¹⁷ is hydrogen or methyl.

Further representative compounds have the formula:

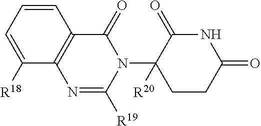

and pharmaceutically acceptable salts, solvates, and stereoisomers thereof, wherein:
R¹⁸ is: hydrogen; halo; —(CH₂)ₙOH; (C₁-C₆)alkyl, optionally substituted with one or more halo; (C₁-C₆)alkoxy, optionally substituted with one or more halo; or

—(CH₂)ₙNHR^{m}, wherein R^{m} is:
hydrogen;
(C1-C6)alkyl, optionally substituted with one or more halo;
—(CH₂)ₙ-(6 to 10 membered aryl);
C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
—C(O)—(C₁-C₈)alkyl, wherein the alkyl is optionally substituted with one or more halo;
—C(O)—(CH₂)ₙ—(C₃-C₁₀-cycloalkyl);
—C(O)—(CH₂)ₙ—NRⁿR^{o}, wherein Rⁿ and R^{o} are each independently:
   hydrogen;
   (C₁-C₆)alkyl, optionally substituted with one or more halo;
   (C₁-C₆)alkoxy, optionally substituted with one or more halo; or
   6 to 10 membered aryl, optionally substituted with one or more of: halo; (C₁-C₆)alkyl, itself optionally substituted with one or more halo; or (C₁-C₆)alkoxy, itself optionally substituted with one or more halo;
—C(O)—(CH₂)ₙ—O—(C₁-C₆)alkyl; or
—C(O)—(CH₂)ₙ—O—(CH₂)ₙ-(6 to 10 membered aryl);
   R¹⁹ is: hydrogen; —(CH₂)ₙOH; phenyl; —O—(C₁-C₆)alkyl; or (C₁-C₆)alkyl, optionally substituted with one or more halo;
   R²⁰ is: hydrogen; or (C₁-C₆)alkyl, optionally substituted with one or more halo; and
   n is 0, 1, or 2.

In one embodiment, R¹⁸ is hydrogen. In another embodiment, R¹⁸ is halo. In another embodiment, R¹⁸ is (C₁-C₆)alkyl, optionally substituted with one or more halo. In another embodiment, R¹⁸ is —(CH₂)ₙOH or hydroxyl. In another embodiment, R¹⁸ is (C₁-C₆)alkoxy, optionally substituted with one or more halo. In one embodiment, R¹⁸ is —(CH₂)ₙNHR^{m}. In one embodiment, wherein R²⁸ is —(CH₂)ₙNHR^{s}, R^{s} is hydrogen. In another embodiment, R^{m} is (C₁-C₆)alkyl, optionally substituted with one or more halo. In another embodiment, R^{m} is —(CH₂)ₙ-(6 to 10 membered aryl). In another embodiment, R^{m} is —C(O)—(CH₂)ₙ-(6 to 10 membered aryl) or —C(O)—(CH₂)ₙ-(6 to 10 membered heteroaryl), wherein the aryl or heteroaryl is optionally substituted as described above. In another embodiment, R^{s} is —C(O)—(C₁-C₈)alkyl, wherein the alkyl is optionally substituted with one or more halo. In another embodiment, R^{m} is —C(O)—(CH₂)ₙ—(C₃-C₁₀-cycloalkyl). In another embodiment, R^{m} is —C(O)—(CH₂)ₙ—NRⁿR^{o}, wherein Rⁿ and R^{o} are as described above. In another embodiment, R^{m} is —C(O)—(CH₂)ₙ—O—(C₁-C₆)alkyl. In another embodiment, R^{m} is —C(O)—(CH₂)ₙ—O—(CH₂)ₙ-(6 to 10 membered aryl).

In one embodiment, R¹⁹ is hydrogen. In another embodiment, R¹⁹ is —(CH₂)ₙOH or hydroxyl. In another embodiment, R¹⁹ is phenyl. In another embodiment, R¹⁹ is —O—(C₁-C₆)alkyl, optionally substituted with one or more halo. In another embodiment, R¹⁹ is (C₁-C₆)alkyl, optionally substituted with one or more halo. In one embodiment, R²⁰ is hydrogen. In another embodiment, R²⁰ is (C₁-C₆)alkyl, optionally substituted with one or more halo. In one embodiment, n is 0. In another embodiment, n is 1. In another embodiment, n is 2. Compounds provided herein encompass any of the combinations of R¹⁸, R¹⁹, R²⁰ and n described above. In one specific embodiment, R¹⁸ is methyl. In another embodiment, R¹⁸ is halo. In another embodiment, R¹⁸ is hydroxyl. In another embodiment, R¹⁸ is —CF₃. In one specific embodiment, R¹⁹ is hydrogen. In another embodiment, R¹⁹ is methyl. In another specific embodiment, R²⁰ is hydrogen.

In another embodiment, provided herein are compounds of formula:

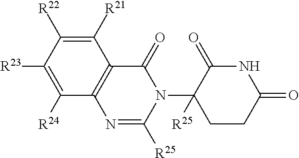

and pharmaceutically acceptable salts, solvates, and stereoisomers thereof, wherein:
R²¹ is hydrogen;
R²², R²³, and R²⁴ are each independently: halo; —(CH₂)ₙOH; (C₁-C₆)alkyl, optionally substituted with one or more halo; (C₁-C₆)alkoxy, optionally substituted with one or more halo; or
two of R²¹-R²⁴ together form a 5 to 6 membered ring, optionally substituted with one or more of: halo; (C₁-C₆)alkyl, optionally substituted with one or more halo; and (C₁-C₆)alkoxy, optionally substituted with one or more halo;
R²⁵ is: hydrogen; —(CH₂)ₙOH; phenyl; —O—(C₁-C₆)alkyl; or (C₁-C₆)alkyl, optionally substituted with one or more halo;
R²⁶ is: hydrogen; or (C₁-C₆)alkyl, optionally substituted with one or more halo; and
n is 0, 1, or 2.

In one embodiment, two of R²²-R²⁴ are halo. In another embodiment, two of R²²-R²⁴ are (C₁-C₆)alkyl, optionally substituted with one or more halo. In another embodiment, two of R²²-R²⁴ are (C₁-C₆)alkoxy, optionally substituted with one or more halo. In another embodiment, one of R²²⁻R²⁴ are is halo, and another one of R²²-R²⁴ is (C₁-C₆)alkyl, optionally substituted with one or more halo. In another embodiment, one of R²²-R²⁴ is halo, and another one of R²²-R²⁴ is (C₁-C₆)alkoxy, optionally substituted with one or more halo. In another embodiment, one of R²²-R²⁴ is (C₁-C₆)alkoxy, optionally substituted with one or more halo, and another one of R²²-R²⁴ is (C₁-C₆)alkyl, optionally substituted with one or more halo. In another embodiment, two of R²²-R²⁴ together form a 5 to 6 membered ring. In one specific embodiment, R²² and R²³ together form a 5 to 6 membered ring. In one specific embodiment, R²² and R²³ together form phenyl ring. In another embodiment, the ring formed by R²² and R²³ is optionally substituted with one or more of: halo; (C₁-C₆)alkyl, optionally substituted with one or more halo; and (C₁-C₆)alkoxy, optionally substituted with one or more halo.

In one embodiment, R²⁵ is hydrogen. In another embodiment, R²⁵ is —(CH₂)ₙOH or hydroxyl. In another embodiment, R²⁵ is phenyl. In another embodiment, R²⁵ is —O—(C₁-C₆)alkyl, optionally substituted with one or more halo. In another embodiment, R²⁵ is (C₁-C₆)alkyl, optionally substituted with one or more halo.

In one embodiment, R²⁶ is hydrogen. In another embodiment, R²⁶ is (C₁-C₆)alkyl, optionally substituted with one or more halo. In one embodiment, n is 0. In another embodiment, n is 1. In another embodiment, n is 2. Compounds provided herein encompass any of the combinations of R²¹, R²², R²³, R²⁴, R²⁵, R²⁶, and n described above.

The compounds provided herein for use also have formula:

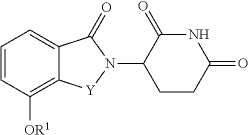

or a pharmaceutically acceptable salt, solvate, prodrug, clathrate, or stereoisomer thereof, wherein Y is C═O or CH₂, and R¹ is hydrogen, alkyl, alkenyl, alkynyl, aryl, aralkyl, cycloalkyl, cycloalkylalkyl, heterocyclyl, heterocyclylalkyl, heteroaryl, heteroarylalkyl, arylaminocarbonyl, alkylcarbonyl, alkylaminocarbonyl, dialkylaminocarbonyl, alkoxycarbonyl, cycloalkylcarbonyl, heteroarylcarbonyl or heterocyclylcarbonyl; where R¹ is optionally substituted with one or more, in certain embodiments, 1, 2, 3 or 4 substituents, one, two or three groups selected from alkoxy, halo, alkyl, carboxy, alkylaminocarbonyl, alkoxycarbonyl, nitro, amine, nitrile, haloalkyl, hydroxy, and alkylsulfonyl.

In one embodiment, Y is C═O. In another embodiment, Y is CH₂.

In certain embodiments, R¹ is alkyl, alkenyl, alkynyl, aryl, aralkyl, cycloalkyl, cycloalkylalkyl, heterocyclyl, heterocyclylalkyl, heteroaryl or heteroarylalkyl, optionally substituted with one or more, in one embodiment, one, two or three groups selected from alkoxy, halo, alkyl and alkylsulfonyl. In one embodiment, R¹ is aryl, aralkyl or heteroarylalkyl. In certain embodiments, the aryl or heteroaryl ring in group R¹ is a 5 or 6 membered monocyclic ring. In certain embodiments, the heteroaryl ring in R¹ group is a 5 or 6 membered monocyclic ring containing 1-3 heteroatoms selected from O, N and S. In certain embodiments, the aryl or heteroaryl ring in group R¹ is a bicyclic ring. In certain embodiments, the heteroaryl ring contains 1-3 heteroatoms selected from O, N and S and is attached to the alkyl group via a hetero atom in the ring. In certain embodiments, the heteroaryl ring is attached to the alkyl group via a carbon atom in the ring. In one embodiment, R¹ is phenyl, benzyl, naphthylmethyl, quinolylmethyl, benzofurylmethyl, benzothienylmethyl, furylmethyl or thienylmethyl, optionally substituted with one or more, in one embodiment, one, two or three groups selected from alkoxy, halo, alkyl and alkylsulfonyl. In one embodiment, R¹ is optionally substituted with one or two substituents selected from methoxy, chloro, bromo, fluoro, methyl and methylsulfonyl. In other embodiments, R¹ is 2-methoxyphenyl, benzyl, 3-chlorobenzyl, 4-chlorobenzyl, 3,4-dichlorobenzyl, 3,5-dichlorobenzyl, 3-fluorobenzyl, 3-bromobenzyl, 3-methylbenzyl, 4-methylsulfonylbenzyl, 3-methoxybenzyl, naphthylmethyl, 3-quinolylmethyl, 2-quinolylmethyl, 2-benzofurylmethyl, 2-benzothienylmethyl, 3-chlorothien-2-ylmethyl, 4-fluorobenzothien-2-ylmethyl, 2-furylmethyl, 5-chlorothien-2-ylmethyl or 1-naphth-2-ylethyl.

In certain embodiments, the compounds have formula:

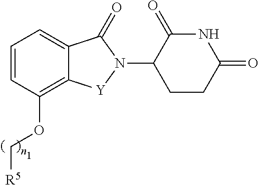

wherein Y is C═O or CH₂, and R⁵ is aryl or heteroaryl, optionally substituted with one, two or three groups selected from alkyl, halo, alkoxy, carboxy, alkylaminocarbonyl, alkoxycarbonyl, nitro, amine, nitrile, haloalkyl, hydroxy, and alkylsulfonyl; n₁ is 0-5, and the other variables are as described elsewhere herein.

In one embodiment, Y is C═O. In another embodiment, Y is CH₂. In one embodiment, n₁ is 0 or 1. In certain embodiments, R⁵ is selected from phenyl, naphthyl, furyl, thienyl, benzofuryl, benzothienyl and quinolyl, optionally substituted with one or two groups selected from methyl, methoxy, chloro, fluoro, bromo and methylsulfonyl. In other embodiments, R⁵ is phenyl, 3-chlorophenyl, 4-chlorophenyl, 3,4-dichlorophenyl, 3,5-dichlorophenyl, 3-fluorophenyl, 3-bromophenyl, 3-methylphenyl, 4-methylsulfonylphenyl, 3-methoxyphenyl, naphthyl, 3-quinolyl, 2-quinolyl, 2-benzofuryl, 2-benzothienyl, 3-chlorothien-2-yl, 4-fluorobenzothien-2-yl, 2-furyl, 5-chlorothien-2-yl or 1-naphth-2-yl.

In another embodiment, the compounds have formula:

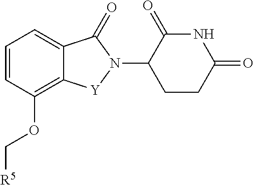

wherein the variables are as described elsewhere herein.

In one embodiment, Y is C═O. In another embodiment, Y is CH₂. In one embodiment, R⁵ is

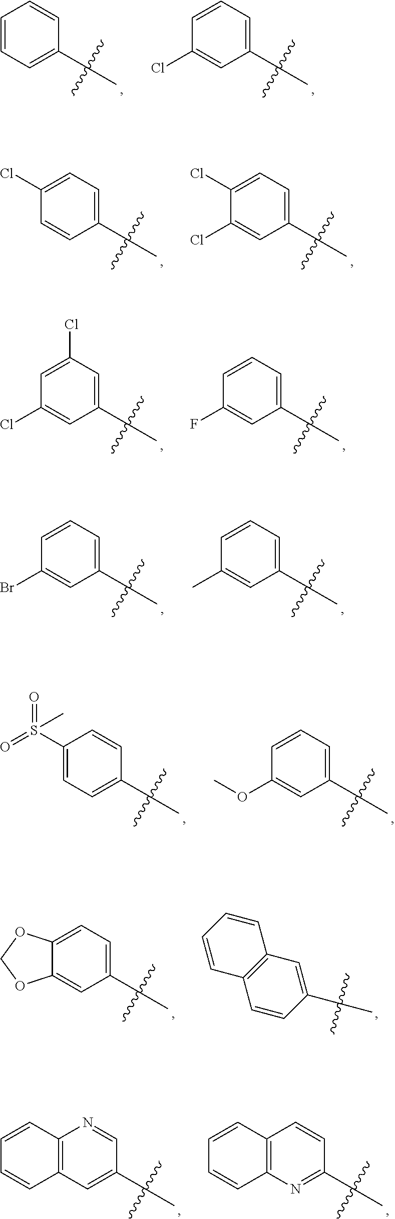

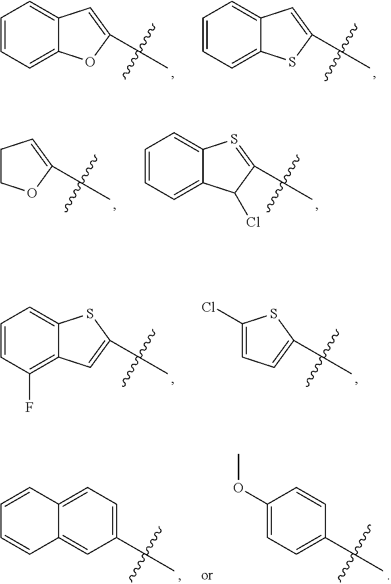

Moreover, as used herein, the compound referred to by the chemical name 3-(2,5-dimethyl-4-oxo-4H-quinazolin-3-yl)piperidine-2,6-dione corresponds to the chemical structure:

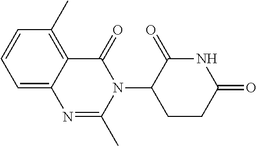 [SMILES: Cc1cccc2nc(C)n(C3CCC(=O)NC3=O)c(=O)c12]

In certain embodiments, the chemical name 3-(2,5-dimethyl-4-oxo-4H-quinazolin-3-yl)piperidine-2,6-dione is used to refer to its free base form or its ionized forms, which have undergone salt formation such that the molecule is protonated at one or more basic centers.

In a specific embodiment, the compound for use herein is 3-(5-amino-2-methyl-4-oxo-4H-quinazolin-3-yl)-piperidine-2,6-dione, which is a Pleiotropic Pathway Modifier (PPM), a novel class of compounds with multiple activities including potent cytokine modulation and antiangiogenic activity, as well as antiproliferative activity. The compound has the following structure:

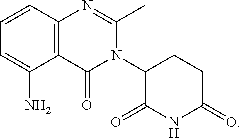 [SMILES: Cc1nc2cccc(N)c2c(=O)n1C1CCC(=O)NC1=O]

The molecular formula is C₁₄H₁₄N₄O₃ and the molecular weight is 286.29. This compound is a 50/50 racemic mixture of a molecule containing one chiral center. This compound is structurally different from IMiD® compounds in that it does not contain the phthalimide/isoindolinone moiety but retains the piperidine-2,6-dione found in IMiD® compounds.

All of the compounds described can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques. Additional information on immunomodulatory compounds, their preparation, and use can be found, for example, in U.S. Patent Application Publication Nos. US20060188475, US20060205787, and US20070049618, each of which is incorporated by reference herein in its entirety.

The compounds may be small organic molecules having a molecular weight less than about 1,000 g/mol, and are not proteins, peptides, oligonucleotides, oligosaccharides or other macromolecules.

In one specific embodiment, the immunomodulatory compound is 4-amino-2-(2,6-dioxopiperidin-3-yl)isoindole-1,3-dione, also known as pomalidomide or Actimid®, having the following structure:

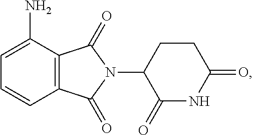 [SMILES: Nc1cccc2c1C(=O)N(C1CCC(=O)NC1=O)C2=O]

or a pharmaceutically acceptable salt, solvate or stereoisomer thereof.

In another embodiment, the immunomodulatory compounds are administered in combination with a second active agent, such as dexamethasone.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 4.5. METHODS OF ADMINISTRATION OF IMMUNOMODULATORY COMPOUNDS

Any route of administration of an immunomodulatory compound may be used. For example, an immunomodulatory compound can be administered by oral, parenteral, intravenous, transdermal, intramuscular, rectal, sublingual, mucosal, nasal, or other means. In addition, an immunomodulatory compounds can be administered in a form of pharmaceutical composition and/or unit dosage form. Suitable dosage forms include, but are not limited to, capsules, tablets (including rapid dissolving and delayed release tablets), powder, syrups, oral suspensions and solutions for parenteral administration. Suitable administration methods for the immunomodulatory compounds, as well as suitable dosage forms and pharmaceutical compositions, can be found in U.S. Patent Application Publication Nos. US20060188475, US20060205787, and US20070049618, each of which is incorporated by reference herein in its entirety.

The specific amount of the agent will depend on the specific agent used, the type of disease or disorder being treated or managed, and the amount(s) of an immunomodulatory compound provided herein and any optional additional agents concurrently administered to the patient. Typical dosage forms comprise an immunomodulatory compound or a pharmaceutically acceptable salt, solvate, stereoisomer, or prodrug thereof in an amount of from about 0.001 to about 150 mg. In particular, dosage forms comprise an immunomodulatory compound or a pharmaceutically acceptable salt, solvate, stereoisomer, or prodrug thereof in an amount of about 0.001, 0.01, 0.1, 1, 2, 5, 7.5, 10, 12.5, 15, 17.5, 20, 25, 50, 100, 150 or 200 mg. In a particular embodiment, a dosage form comprises 4-(amino)-2-(2,6-dioxo(3-piperidyl))-isoindoline-1,3-dione in an amount of about 0.1, 0.2, 0.5, 1.0, 2.0, 2.5, 3.0, 4.0, 5.0, or 10 mg.

Pharmaceutical compositions provided herein can also contain one of more pharmaceutically acceptable excipients. See, e.g., Rowe et al., Handbook of Pharmaceutical Excipients, 4^{th} Ed. (2003), entirety of which is incorporated herein by reference.

In some embodiments, an immunomodulatory compound is administered to a subject about 3 months, 30 days, 20 days, 15 days, 12 days, 10 days, 7 days, 5 days, 3 days, 1 day, 12 hours, or 5 hours prior to testing for protein biomarker levels. In other embodiments, an immunomodulatory compound is administered from about 3 months to about 30 days, 30 days to about 5 hours, from about 20 days to about 5 hours, from about 15 days to about 12 hours, from about 12 days to about 5 hours, from about 10 days to about 12 hours, from about 7 days to about 12 hours, from about 5 days to about 12 hours, from about 5 days to about 1 day, from about 3 days to about 12 hours, or from about 3 days to about 1 day prior to testing for protein biomarker levels.

In some embodiments, provided herein is protein biomarker-based monitoring upon administration of racemic mixture, optically pure (R)-isomer, or optically pure (S)-isomer of 4-(amino)-2-(2,6-dioxo(3-piperidyl))-isoindoline-1,3-dione. In one specific embodiment, the racemic 4-(amino)-2-(2,6-dioxo(3-piperidyl))-isoindoline-1,3-dione is administered at an amount of 0.5 to 4 mg per day. As (S)-isomer of 4-(amino)-2-(2,6-dioxo(3-piperidyl))-isoindoline-1,3-dione is reported to have a higher potency than the racemic mixture, a lower dose can be given when (S)-isomer is used. For example, (S)-4-(amino)-2-(2,6-dioxo(3-piperidyl))-isoindoline-1,3-dione can be administered at an amount of 0.01, 0.1, 1, 2.5, 5, or 10 mg per day. The (R)-isomer of 4-(amino)-2-(2,6-dioxo(3-piperidyl))-isoindoline-1,3-dione can be administered at an amount comparable to the racemic mixture.

In a specific embodiment, a dosage form comprises 3-(4-amino-1-oxo-1,3-dihydro-isoindol-2-yl)-piperidine-2,6-dione in an amount of about 5, 10, 15 or 25 mg. Also provided herein is the use of racemic mixture, (S)-isomer, and (R)-isomer of 3-(4-amino-1-oxo-1,3-dihydro-isoindol-2-yl)-piperidine-2,6-dione. Typically, racemic 3-(4-amino-1-oxo-1,3-dihydro-isoindol-2-yl)-piperidine-2,6-dione can be administered at an amount of 1, 5, 10, 15, 25, or 50 mg per day. Optical isomers also can be administered at an amount comparable to racemic mixture. Doses can be adjusted depending on the type of disease or disorder being treated, prevented or managed, and the amount of an immunomodulatory compound and any optional additional agents concurrently administered to the patient, which are all within the skill of the art.

In certain embodiments, pomalidomide is administered in an amount of from 0.1 to about 10 mg per day. In certain embodiments, pomalidomide is administered in an amount of about 0.5 mg to about 4 mg per day. In certain embodiments, the compound is administered in an amount of about 2 mg per day. In certain embodiments, the compound is administered cyclically. In certain embodiments, the cycle comprises four weeks. In other embodiments, the cycle comprises the administration of the compound for 21 days followed by seven days rest. In certain embodiments, the compound is administered in an amount of from about 0.5 mg to about 4 mg per day for 21 days followed by seven days rest in a 28 day cycle.

As described herein, the immunomodulatory compounds can be administered in combination with a therapeutically effective amount of a second active agent. In certain embodiments, the second active agent is dexamethasone. In certain embodiments, dexamethasone as a second active agent is administered in an amount of about 40 mg once daily on days 1 to 4, 9 to 12, and 17 to 20 every 28 days. In certain embodiments, dexamethasone as a second active agent is administered in an amount of about 40 mg once daily on days 1, 8, 15, and 22 every 28 days.

In certain embodiments, pomalidomide is orally administered in an amount of from about 0.5 mg to about 2 mg per day on days 1 through 28 every 28 days, and dexamethasone is administered in an amount of about 40 mg once daily on days 1, 8, 15, and 22 every 28 days. In certain embodiments, the compound is administered orally, which may be in the form of a capsule or tablet.

### 5. EXAMPLES

The examples below are carried out using standard techniques, which are well known and routine to those of skill in the art, except where otherwise described in detail. The examples are intended to be merely illustrative.

### 5.1 METHODS

In order to support pomalidomide development decisions and registration, a modeling framework is developed as described herein, which allows dose-response simulations of clinical endpoints in refractory multiple myeloma patient following treatment with pomalidomide in combination with dexamethasone. Change in serum M-protein concentration, a marker for tumor burden, is taken as a biomarker of drug effect. Change in serum M-protein level can be used as a predictor of clinical endpoints of interest (e.g. survival or PFS) in an approach similar to solid tumors (see Claret et al., J. Clin. Oncol. (2009), 27: 4103-8; Wang et al., Clin. Pharmacol. Ther. (2009), 86: 167-74).

Thus, longitudinal tumor growth inhibition models for the time course of serum M-protein measurements (taken as a marker of tumor size) were developed for therapy with dexamethasone and pomalidomide. The models account for tumor growth, exposure (dose) driven drug effect and resistance appearance. The models were qualified to simulate change from baseline in M-protein at the end of cycle 2 (week 8).

5.1.1 Trials and Data Sources

### Lenalidomide Studies

M-protein measurements were derived from dexamethasone data obtained from 704 patients included in two historical Phase III clinical trials of lenalidomide plus dexamethasone vs. dexamethasone alone (Dimopoulos et al., New Engl. J. Med. (2007), 357: 2123-32; Weber et al., New Eng. J. Med. (2007), 357: 2133-42). Data from 704 patients randomized in the two Phase III studies (353 and 351 respectively) and 222 patients included in an additional lenalidomide monotherapy multicenter, single-arm, open-label Phase II study of subjects with relapsed and refractory multiple myeloma (Richardson et al., Blood (2005) 106: 449a, Abstract #1565) were delivered in SAS transport format, read in and manipulated using SPLUS 8.1 (Insightful) and output to a NONMEM-readable ASCII file.

### Pomalidomide Studies

M-protein measurements entered from data with pomalidomide in relapsed and refractory multiple myeloma patients consists of one completed and two ongoing studies. In a Phase Ib ascending dose study in 45 patients, the MTD of pomalidomide was 2 mg administered daily or 5 mg administered on an every other day schedule. These subjects tolerated alternate day dosing better than daily dosing based on dose-limiting toxicity (DLT), however, the AE profiles were similar for both dosing regimens.

Two clinical studies are ongoing in the same patient population: A Phase I/II, multicenter, randomized, open-label, dose-escalation study evaluates the safety and efficacy of pomalidomide alone and in combination with oral dexamethasone in patients (up to 212) with relapsed and refractory multiple myeloma in ongoing. This study consists of a Phase I single-agent dose-finding segment and a randomized segment (pomalidomide plus dexamethasone versus pomalidomide alone) where pomalidomide is administered once daily on days 1-21 of each 28-day cycle (cyclic regimen schedule). The Phase I segment investigates whether pomalidomide single agent dose could be escalated up to 5 mg daily using the cyclic schedule. The Phase II segment has progression free survival (PFS) as the primary endpoint and an interim analysis is planned at 50% of the events. Oral dexamethasone 40 mg is administered on Days 1, 8, 15, and 22 of each 28-day treatment cycle.

The study data (SAS files) were read in and manipulated using SPLUS 8.0 (Insightful) and output to a NONMEM-readable ASCII file in two different data sets delivered Mar. 3, 2010 (76 patients) and Nov. 17, 2010 (217 patients).

5.1.2 Tumor Growth Inhibition Model

A semi-mechanistic exposure-driven tumor growth inhibition (TGI) model was used to model serum M-protein data (taken as a marker of tumor size) as function of time and drug dose levels. The TGI model is described by the following differential equation system:

$$\frac{d y ( t )}{d t} = {K}_{L} \cdot y ( t ) - {K}_{D} ( t ) \cdot D ( t ) \cdot y ( t )$$

$${K}_{D} ( t ) = {K}_{D , 0} \cdot {e}^{- λ t}$$

$$\frac{d D ( t )}{d t} = - {K}_{P} \cdot D ( t )$$

$$y ( 0 ) = {y}_{0}$$

where y(t) is the at time t with y₀ value at baseline (g/L),

K_{L}, is the rate of M-protein level increase (tumor growth rate) (week⁻¹),

K_{D}, is the rate of drug-induced M-protein decrease (drug potency, mg⁻¹.week⁻¹)

K_{D} decreases with time (from K_{D,0} at time 0, full effect) with the rate constant λ, (week⁻¹)

D (t) is the amount of drug at the site of action (mg) (“KPD model”) (see Jacqim et al., J. Pharmacokinet. Pharmacodyn. (2007), 31: 57-85),

Kₚ is the elimination rate constant from the virtual biophase compartment (week⁻¹).

Inter-patient variability of parameters was modeled using exponential random effects as follows:

θᵢ=θ·e^{ηi}

ηᵢ→N(0,ω²

where 0, is the individual parameter of the j^{th} individual,

θ is the typical (population mean) value of the parameter and

ηᵢ denotes the normally distributed inter-patient random effect accounting for the

i^{th} individual's with zero mean and variance ω².

No covariance between the random effects was considered (diagonal covariance matrix).

Residual variability was modeled using a combination of proportional and additive errors and residual random effects (ε₁ᵢⱼ and ε₂ᵢⱼ for patient i at time j) normally distributed with mean zero and variance vector σ² (comprising σ₁² and σ₁²).

y_{obs,i}(tⱼ)=yᵢ(tⱼ)·(1+ε_{2,ij})+ε_{1,ij}

εᵢⱼ→N(0,σ²)

where y_{obs,i} is the observed M-protein level for patient I and tⱼ is the observation time.

Model parameters were estimated by maximum likelihood in non-linear mixed effect model using NONMEM VI level 1.0 FOCE method with interaction (Beal et al., NONMEM user's guide. (1992) San Francisco: University of California at San Francisco NONMEM Project Group).

Nested models were compared using the likelihood ratio test in which the objective function (−2 log likelihood (−2LL)) of a full model (i.e. a model with study effect on a given parameter) is compared to that of a reduced model (i.e. a model without the study effect). The difference (6) in log likelihood of the two models is asymptotically X² distributed with q degrees of freedom where q is the difference between the number of parameters in the full model and in the reduced model.

Goodness of fit of different models to the data was evaluated using the following criteria: change in the objective function, visual inspection of different diagnostic plots, precision of the parameter estimates. Diagnostic plots examined to assess model adequacy, possible lack of fit or violation of assumptions were: Observed (DV) vs. population predicted (PREDICTED) or individual predicted values (I.PREDICTED) values with line of unity.

The TGI model was subject to an internal simulation-based evaluation using a posterior predictive check (PPC). PPC uses the model and the study design to simulate statistics of interest (median and quartiles of fractional change in M-protein at week 8) of many (hypothetical) trial replicates (n=500) across model parameter uncertainty (for different replicates), inter-individual variability (within replicates) and residual error. If the observed trial statistics fall within the predictive distribution of the simulated statistics, the model is qualified.

5.1.3 Survival Models

### Exploratory Data Analysis

Overall survival and PFS data were explored using Kaplan-Meier and Cox regression analyses using survfit( ) and coxph( ) functions, respectively in S-plus version 8.0. A number of baseline characteristics together with individual predictions of change in serum M-protein from baseline at first post-treatment visit (week 8) (a measure of drug effect) were assessed one by one in the Cox model.

Individual predictions of change in serum M-protein from baseline were obtained using an empirical model similar to the one proposed by FDA scientists to analyze longitudinal non-small cell lung cancer tumor size data (Wang et al., Clin. Pharmacol. Ther. (2009), 86: 167-74).

### Parametric Survival Models

Parametric survival models were developed for overall survival and PFS. The models describe the survival time distribution as a function of covariates. The probability density function that best described the observed survival time was selected among normal, lognormal, Weibull, logistic, loglogistic, exponential and extreme using difference in log-likelihood of the alternative models.

Model parameter estimation was done using the Censor Reg function in S-plus version 8.0. Of note, the survival model can be considered as a drug-independent model relating a biomarker response (i.e. change in M-protein) and prognostic factors (e.g. baseline M-protein and albumin levels, prior therapies) to a clinical endpoint (survival time or PFS time).

The covariates significant in the univariate Cox model were tested and removed one per one. Covariates with p<0.01 were kept for next step evaluation.

The survival models were subject to both internal and external evaluations: Internal evaluation used a PPC: Survival times for the same number of patients as in the pooled dataset (MM-009 and MM-010) were simulated 1000 times. Parameter values for the survival and PFS models were sampled from the estimated mean values and variance-covariance matrix (uncertainty in parameter estimates). Simulated survival and PFS distribution were compared to observed. If observed distribution falls within the 95% prediction interval, the model is qualified. External evaluation consisted in simulating multiple replicates of an independent study (see 5.1) such as described below, and compare simulated distributions to observed.

### Simulations

The final tumor size, survival and PFS models were used to simulate multiple replicates of pomalidomide study outcome as follow:
Predicted relative change at end of cycle 2 of serum M-protein and study patients characteristics were used to predict patients PFS and survival.
Study simulations were replicated a large number of times (10000 replicates) in including PFS and survival model parameter uncertainties at replicate level.
Predicted patient simulated PFS and survival were subjected to Kaplan Meier analysis at replicate level.
Expected (and 95% CI) PFS and survival KM curves were computed across replicates.

### 5.2 TUMOR GROWTH INHIBITION MODEL FOR DEXAMETHASONE

5.2.1 Exploratory Data Analysis

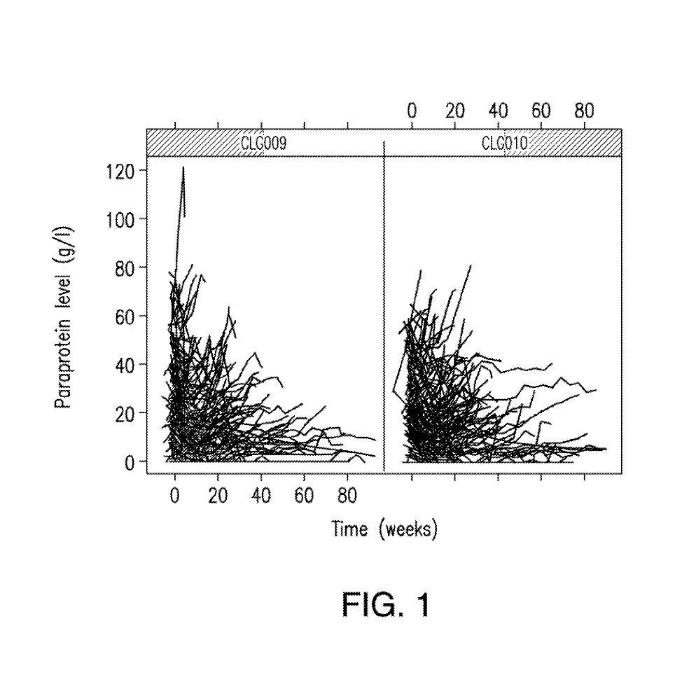

Overall, 2422 observations were available for 346 patients (7 measurements per patient) out of the 351 entered in the dexamethasone (placebo) arms of studies MM-009 and MM-010. To be evaluable in this analysis, patients needed to have at least one M-protein measurement and available dosing records in the data set. In addition, it was required that the patient should have at least one M-protein measurement before the first dose. The M-protein longitudinal profiles are illustrated in FIG. 1 (note the solid line is a smooth of the data).

5.2.2 TGI Model Development

Data did not support estimation of K_{P}, the elimination rate constant from virtual biophase. A log-likelihood profile involving multiple runs for a plausible range of K_{P} values was created in order to determine the optimal one given the data: a value of 20 was selected. Parameter estimates are given in Table I.

TABLE I
Dexamethasone TGI model parameter estimates

| | | | Interindividual | |
|---|---|---|---|---|
| Parameter | Estimate | RSE (%)* | variability ω | RSE* (%) |
| K_{L} (wk⁻¹) | 0.0264 | 8.7 | 0.87 | 15 |
| K_{D} (mg⁻¹wk⁻¹) | 0.0265 | 6.0 | 0.67 | 13 |
| λ (wk⁻¹) | 0.158 | 8.9 | 0.58 | 34 |
| σ₁ (g/L) | 1.90 | 19 | | |
| σ₂ (%) | 0.101 | 33 | | |

ω: standard deviation in log scale (approximate CV).
RSE: relative standard error of parameter estimates

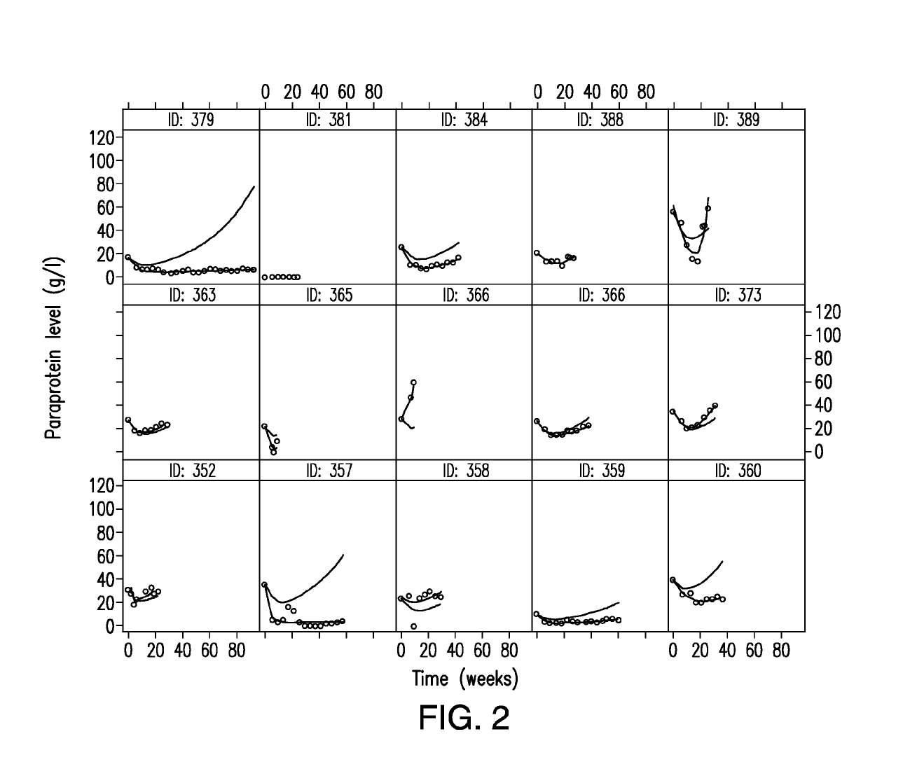

All structural parameters are well estimated with relative SE lower than 10%. Residual variability is low (1.90 g/L for the additive part and 10% for the proportional one). The tumor growth doubling time (based on M-protein) is 26 weeks for the typical patient with a large inter-patient variability. The model provides a satisfactory fit of the data as illustrated in FIG. 2 for a sample of individual patients.

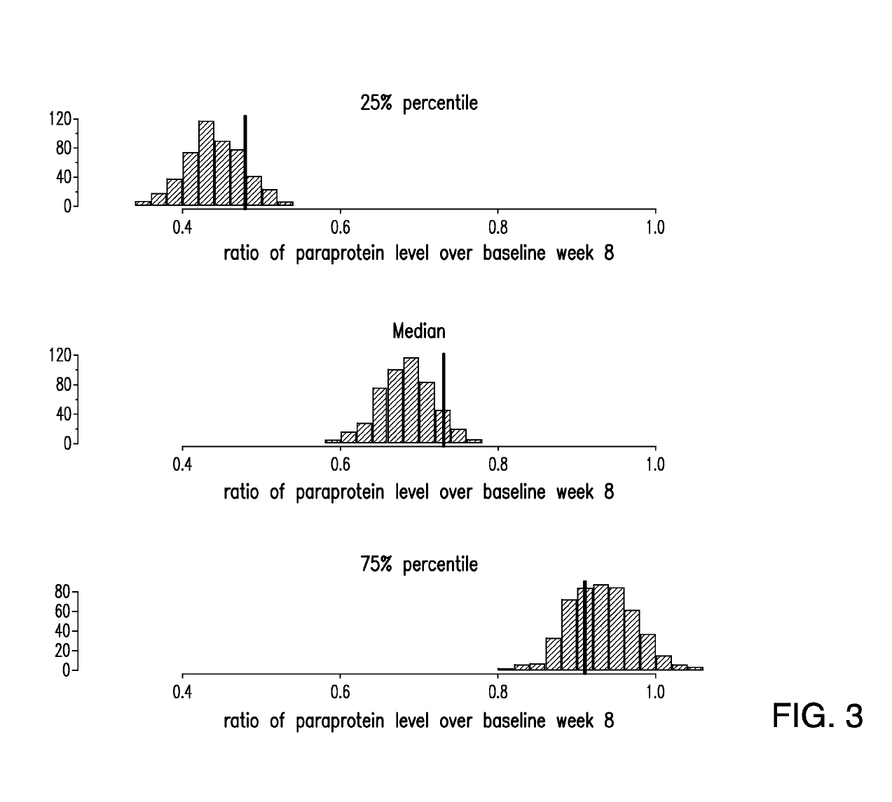

The posterior predictive check shown in FIG. 3 indicates acceptable performance of the model in simulating fractional change in M-protein at week 6 (note that the statistics are medians and quartiles of fractional change in M-protein at week 8 across 500 replicates, vertical lines are observed). This could be demonstrated not only for the median but also for the quartiles (Q25% and Q75%). The model is qualified to simulate relative change of M-protein level at end of cycle 2 (week 8).

### 5.3 TUMOR GROWTH INHIBITION MODEL FOR POMALIDOMIDE

5.3.1 Exploratory Data Analysis

As some of these subjects in the underlying study received dexamethasone in addition to pomalidomide (either after progression in patient receiving pomalidomide single agent or in the Phase II combination arm), data were censored after start of dexamethasone treatment.

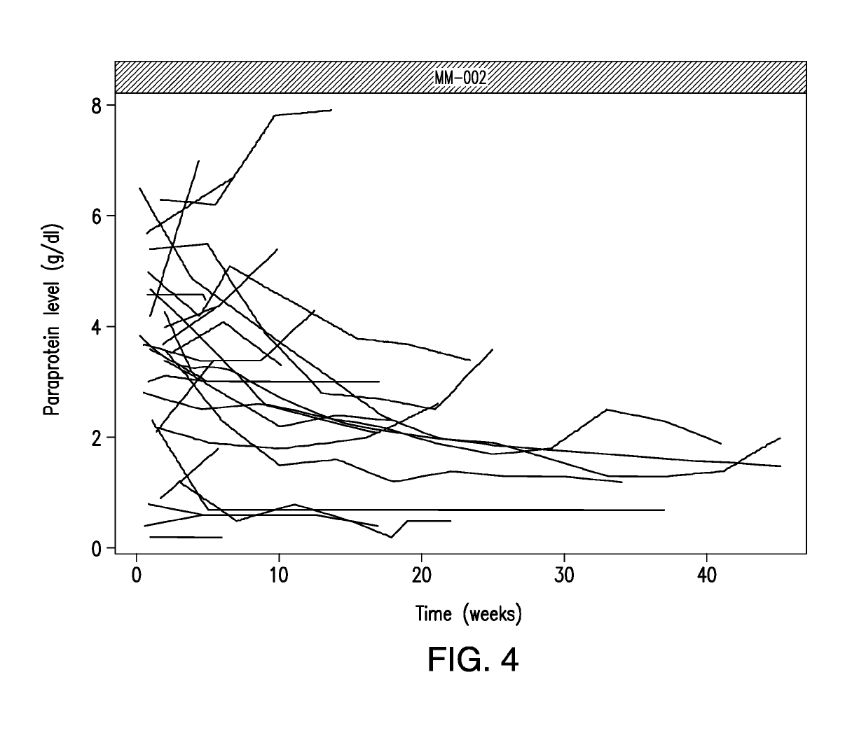

Overall, 130 observations were available for 37 patients (3.5 measurements per patient): 28 entered in the Phase I part and 9 entered in the Phase II part of the study out of the 76 patient in the dataset. To be evaluable in this analysis, patients needed to have at least one M-protein measurement, dosing records in the data set and have been receiving pomalidomide single agent. M-protein longitudinal profiles are illustrated in FIG. 4 (note the solid line is a smooth of the data).

5.3.2 TGI Model Development

Data did not support estimation of K_{P}, the elimination rate constant from virtual biophase. A log-likelihood profile involving multiple runs for a plausible range of K_{P} values was created in order to determine the optimal one given the data: a value of 10 was selected. Parameter estimates are given in Table II.

TABLE II
Pomalidomide TGI model parameter estimates

| | | | Interindividual | |
|---|---|---|---|---|
| Parameter | Estimate | RSE (%) | variability ω | RSE (%) |
| K_{L} (wk⁻¹) | 0.036 | 36.1 | 0.83 | 24.1 |
| K_{D} (mg⁻¹wk⁻¹) | 0.198 | 28.8 | 0.94 | 26.6 |
| λ (wk⁻¹) | 0.128 | 13.3 | | |
| σ₁ (g/dL) | 0.28 | 10.0 | | |

ω: standard deviation in log scale (approximate CV). Couldn't be estimated for λ.
RSE: relative standard error of parameter estimates

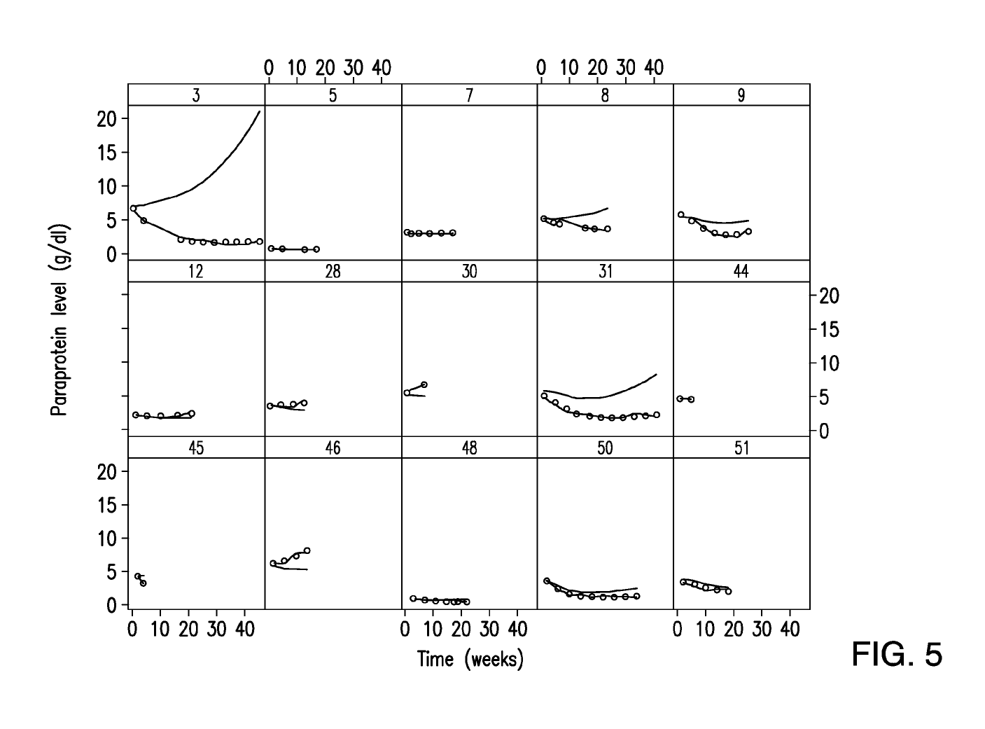

All structural parameters are well estimated with relative SE lower than 10%. Residual variability is low (0.28 g/dL for the additive part). The tumor growth doubling time (based on M-protein) is 19 weeks for the typical patient with a large inter-patient variability. The model provides a satisfactory fit of the data as illustrated in FIG. 5 for individual patients.

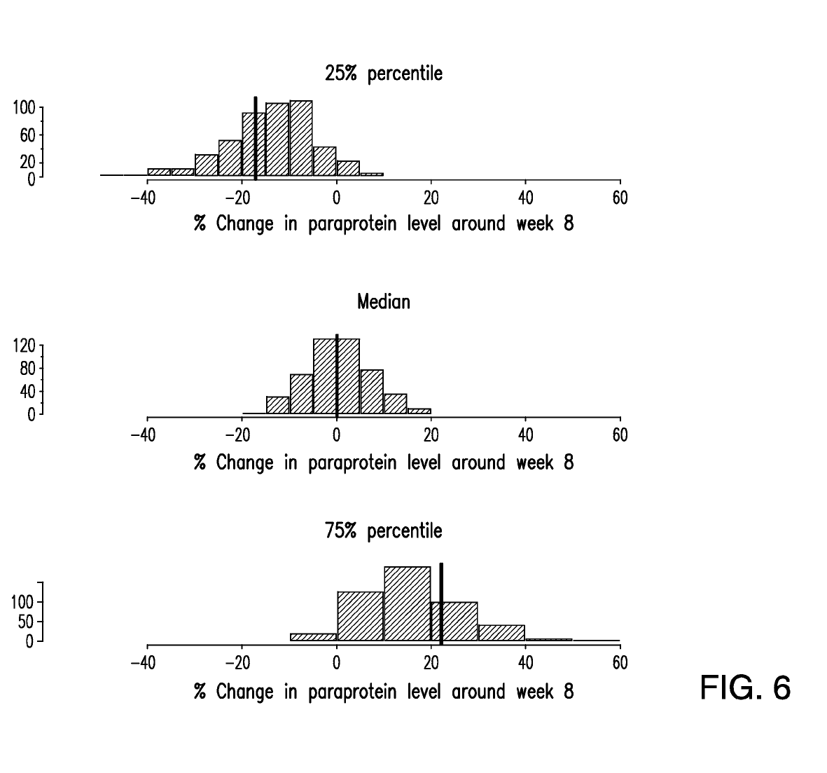

The posterior predictive check shown in FIG. 6 indicates acceptable performance of the model in simulating fractional change in M-protein at week 8 (actually week 7-12 as only 7 patients had M-protein measurements at week 8). Also note that the statistics are medians and quartiles of fractional change in M-protein at week 8 across 500 replicates, vertical lines are observed. Only 7 patients had M-protein values at 8 weeks. The number of patients increased to 27 by considering weeks 4-12 instead. This could be demonstrated not only for the median but also for the quartiles (Q25% and Q75%). The model is qualified to simulate relative change of M-protein level at end of cycle 2 (week 8).

The tumor growth inhibition models for dexamethasone (5.2) and for pomalidomide (5.3) link drug dose intensity to the effect on serum M-protein taken as a marker of tumor size. These models support dose and schedule determination for pomalidomide single agent or in combination with dexamethasone and selection of a 4 mg dose once a day using a cyclic regimen (21 of 28 days) and in combination with low-dose dexamethasone for clinical evaluation.

### 5.4 SURVIVAL MODEL

5.4.1 Exploratory Data Analysis

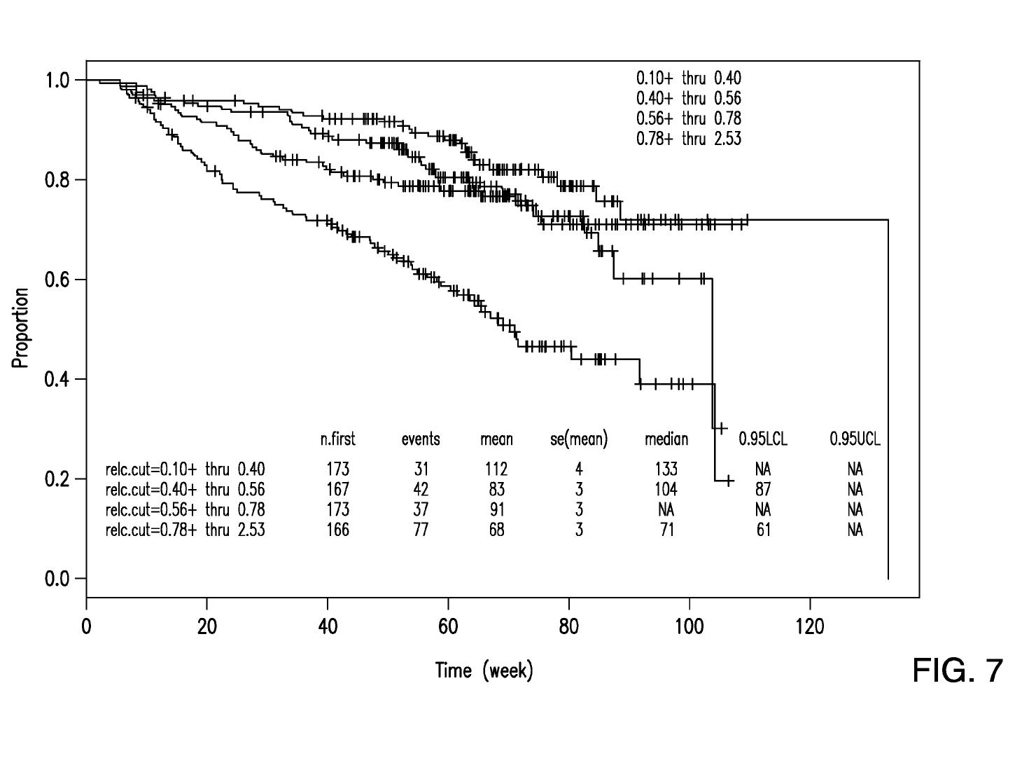

Among the 704 patients included in the 2 Phase III studies, 679 (96.5%) were evaluable for survival modeling. To be evaluable, patients needed to have at least two M-protein measurements to be analyzed using the empirical M-protein model to predict end-of-cycle 2 (week 8) change from baseline in M-protein. Among these patients only 187 (27.5%) died during the observation time with clear survival difference between the two treatments (not shown), by quartiles of baseline M-protein (not shown) and by quartiles in M-protein change from baseline at week 8 (FIG. 7).

Exploratory Cox regression analyses indicated that a number of covariates significantly impacted survival including baseline and change from baseline M-protein level (Table III).

TABLE III
Cox regression exploratory analysis for survival

| Covariate | P value |
|---|---|
| Treatment | 0.00041 |
| Study | 0.05423 |
| Sex (M/F) | 0.18780 |
| Age | 0.00068 |
| Albumin (g/L) | 0.00000 |
| Beta-2M (mg/L) | 0.05093 |
| Calcium (mmol/L) | 0.90429 |
| Creatinine (umol/L) | 0.00000 |
| ECOG > 1 | 0.00000 |
| Hemoglobin (g/L) | 0.00000 |
| MM duration | 0.05656 |
| Prior dexamethasone (Yes/No) | 0.50693 |
| Prior doxorubicin (Yes/No) | 0.52084 |
| Prior melphalan (Yes/No) | 0.02096 |
| Number of prior stem cell transplant | 0.33509 |
| Prior thalidomide (Yes/No) | 0.16279 |
| Prior bortezomib (Yes/No) | 0.23280 |
| Worsening lytic bone disease (Yes/No) | 0.03024 |
| Number of lytic bone lesions | 0.06539 |
| MM stage | 0.06444 |
| Lytic bone lesions (absent/present) | 0.15843 |
| Serum M-protein at baseline (pred.) | 0.00000 |
| Serum M-protein change from baseline (pred.) | 0.00000 |

5.4.2 Parametric Survival Model

The lognormal distribution had the best likelihood (Table IV) and was selected for further analyses.

TABLE IV
Log-likelihood for different survival time distributions

| Distribution | Log-likelihood |
|---|---|
| Weibull | 2365.8 |
| Lognormal | 2363.9 |
| Loglogistic | 2366.5 |
| Extreme | 2479.9 |
| Normal | 2447.3 |
| Logistic | 2468.6 |

After stepwise deletion of the covariates significant in the Cox model, the final model included:

Predicted M-protein change from baseline at week 8

ECOG Performance status

Baseline albumin level

Baseline creatinine level

Baseline hemoglobin level.

Parameter estimates for the final model are given in Table V.

TABLE V
Parameter estimates for the final survival model

| | Estimate | SE | z | p |
|---|---|---|---|---|
| Intercept | 3.317 | 0.5522 | 6.01 | 1.90E−09 |
| ECOG | −0.648 | 0.1941 | −3.34 | 8.48E−04 |
| Albumin | 0.464 | 0.1351 | 3.43 | 5.99E−04 |
| Hemoglobin | 0.108 | 0.0411 | 2.63 | 8.44E−03 |
| Creatinine | −0.582 | 0.1786 | −3.26 | 1.12E−03 |
| M-potein cfb | −0.996 | 0.1583 | −6.29 | 3.16E−10 |
| Log (scale) | 0.202 | 0.0592 | 3.41 | 6.56E−04 |

Note:
SE: standard error of parameter estimates,
z: z statistic,
p: p value for z

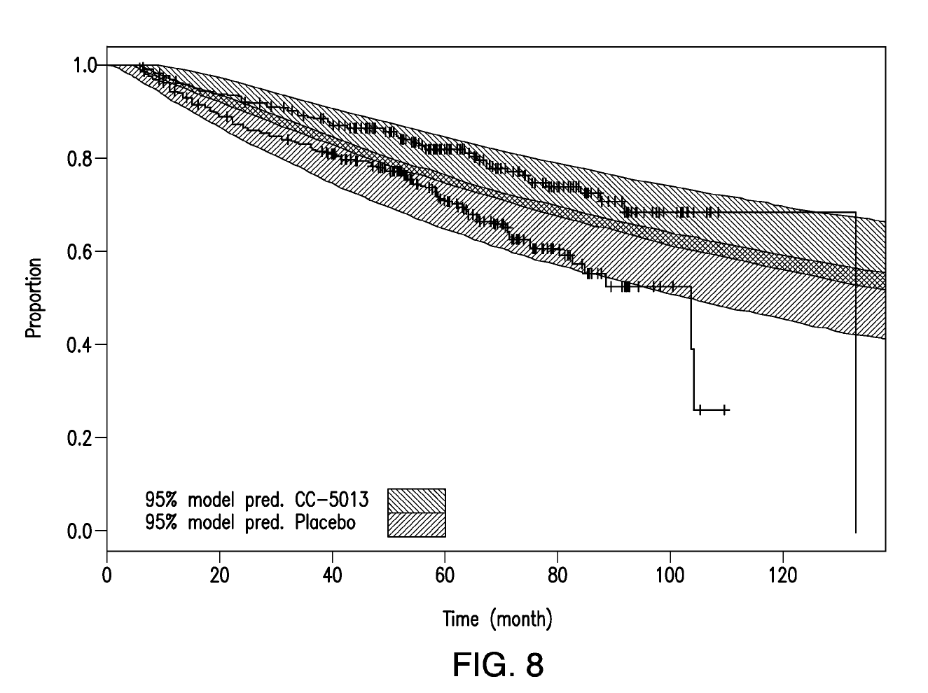

In this model, survival decreases in patients with EGOG>1, with decreased baseline albumin and hemoglobin, increased baseline creatinine, and increased M-protein change from baseline (tumor progression). Of interest, treatment effect that was strongly significant in univariate Cox analysis is no longer in the final model indicating that change in M-protein from baseline fully captured treatment difference. The posterior predictive check indicated good performance of the model in simulating survival in the pooled data (not shown) and in simulating treatment difference (FIG. 8).

### 5.5 PROGRESSION-FREE SURVIVAL

5.5.1 Exploratory Data Analysis

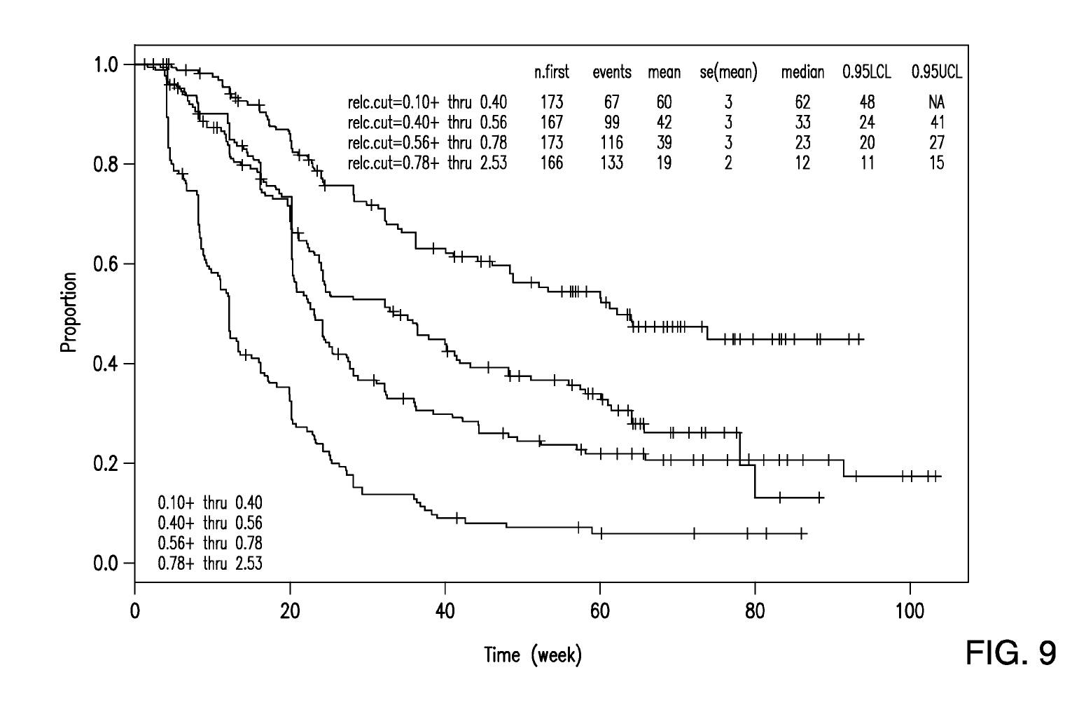

The PFS analysis was performed in the same patient population as the survival one (679 patients). Among these patients 416 (61.2%) had an event during the observation time with large PFS difference between the two treatments (not shown), by quartiles of baseline M-protein (not shown) and by quartiles in M-protein change from baseline at week 8 (FIG. 9).

Exploratory Cox regression analyses indicated that a number of covariates significantly impacted survival including baseline and change from baseline M-protein level (Table VI).

TABLE VI
Cox regression exploratory analysis for PFS

| Covariate | P value |
|---|---|
| Treatment | 0.00000 |
| Study | 0.51484 |
| Sex (M/F) | 0.81795 |
| Age | 0.15442 |
| Albumin (g/L) | 0.00000 |
| Beta-2M (mg/L) | 0.13710 |
| Calcium (mmol/lL) | 0.48736 |
| Creatinine (umol/L) | 0.00118 |
| ECOG>1 | 0.43878 |
| Hemoglobin (g/L) | 0.00000 |
| MM duration | 0.23374 |
| Prior dexamethasone (Yes/No) | 0.15667 |
| Prior doxorubicine (Yes/No) | 0.07412 |
| Prior melphalan (Yes/No) | 0.61963 |
| Number of prior stem cell transplant | 0.05654 |
| Prior thalidomide (Yes/No) | 0.00046 |
| Prior velcade (Yes/No) | 0.26481 |
| Worsening lytic bone disease (Yes/No) | 0.53277 |
| Number of lytic bone lesions | 0.76592 |
| MM stage | 0.44564 |
| Lytic bone lesions (absent/present) | 0.89204 |
| Serum M-protein at baseline (pred.) | 0.00000 |
| Serum M-protein change from baseline (pred.) | 0.00000 |

5.5.2 Parametric Survival Model for PFS

The lognormal distribution had the best likelihood (Table VII) and was selected for further analyses.

TABLE VII
Log-likelihood for different PFS time distributions

| Distribution | Log-likelihood |
|---|---|
| Weibull | 4009 |
| Lognormal | 3932 |
| Loglogistic | 3946 |
| Extreme | 4577 |
| Normal | 4377 |
| Logistic | 4387 |

After stepwise deletion of the covariates significant in the Cox model, the final model included:

Predicted M-protein change from baseline at week 8

Baseline hemoglobin level

Treatment.

In this model, treatment effect is still significant indicating that change in M-protein from baseline did not fully capture PFS treatment difference as opposed as was observed in the survival model. There was no significant interaction between treatment and change in M-protein. The posterior predictive check indicated acceptable performance of the model in simulating in simulating PFS treatment difference but when the treatment effect was omitted, treatment difference was under-estimated. Separate models for the two treatments were finally developed. The same covariates were significant in the model and parameter estimates for the final models are given in Table VIII.

TABLE VIII
Parameter estimates for the final PFS models

| | Estimate | SE | z | p |
|---|---|---|---|---|
| Dexamethasone | | | | |
| Intercept | 2.248 | 0.2932 | 7.67 | 1.78E−14 |
| M-protein cfb | −1.1 | 0.0999 | −11.02 | 3.13E−28 |
| Hemoglobin | 0.123 | 0.023 | 5.37 | 8.07E−08 |
| Log (scale) | −0.338 | 0.0455 | −7.42 | 1.14E−13 |
| Combination | | | | |
| Intercept | 3.1634 | 0.4733 | 6.68 | 2.34E−11 |
| M-protein cfb | −1.6194 | 0.3515 | −4.61 | 4.08E−06 |
| Hemoglobin | 0.1245 | 0.0388 | 3.21 | 1.34E−03 |
| Log (scale) | 0.0935 | 0.0623 | 1.5 | 1.33E−01 |

Note:
SE: standard error of parameter estimates,
z: z statistic,
p: p value for z

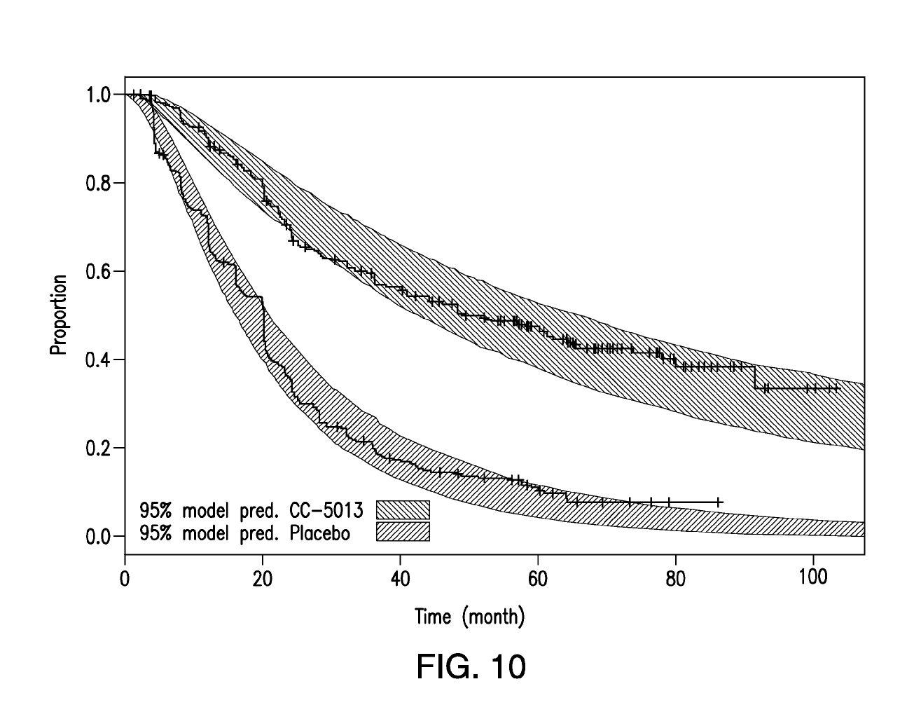

In these models, PFS decreases in patients with decreased baseline hemoglobin and increased M-protein change from baseline (tumor progression). The posterior predictive check indicated good performance of the model in simulating treatment difference (FIG. 10).

Thus two models were also developed for survival (5.4) and PFS (5.5) linking change in M-protein from baseline at the end of cycle 2—i.e. week 8—(taken as a biomarker of drug effect) and prognostic factors to survival and PFS times. The survival model is drug-independent, i.e. end of cycle 2 M-protein response is fully capturing treatment effect at least for lenalidomide and dexamethasone. Specific models were developed for dexamethasone and lenalidomide plus dexamethasone. The models were qualified to simulate survival and PFS distributions in both the studies that were used to build the models and an independent study.

### 5.6 SIMULATIONS

5.6.1 Simulations of Lenalidomide Clinical Trial

The survival and PFS models were evaluated in simulating the single arm Phase II lenalidomide trial MM-014 (external evaluation of the models). To be evaluable, patients needed to have at least two M-protein measurements to be analyzed using the empirical M-protein model to predict end-of-cycle 2 (week 8) change from baseline in M-protein and non-missing model covariates:
205 out of 222 (92%) entered in the study patients with predictable serum M and observed hemoglobin
191 out of 222 (86%) entered in the study patients with predictable serum M and observed albumin, hemoglobin and ECOG.

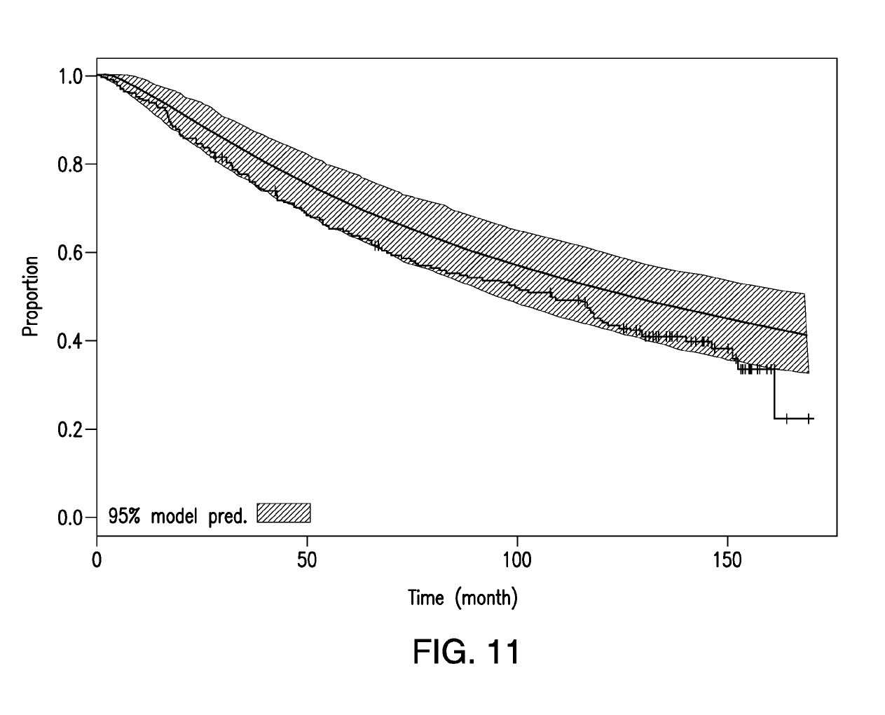

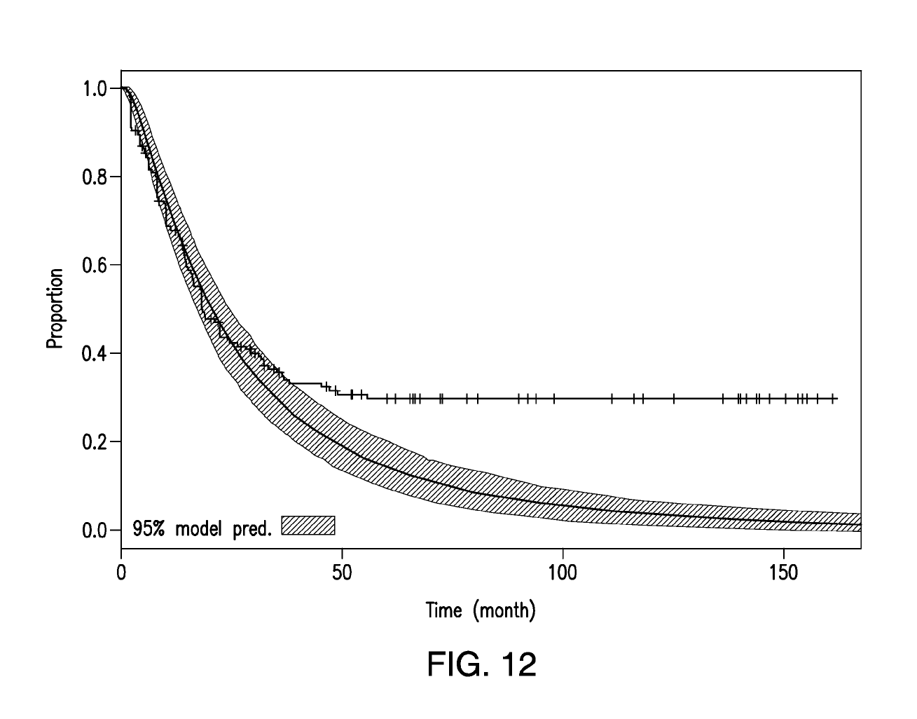

The empirical FDA tumor size model (Wang et al., Clin. Pharmacol. Ther. (2009), 86: 167-74) provided a good fit of M-protein data. Simulations illustrated in FIG. 11 (survival) and FIG. 12 (PFS) indicate good performance in the model to simulate results from this independent study.

5.6.2 Simulations of Pomalidomide Clinical Trial

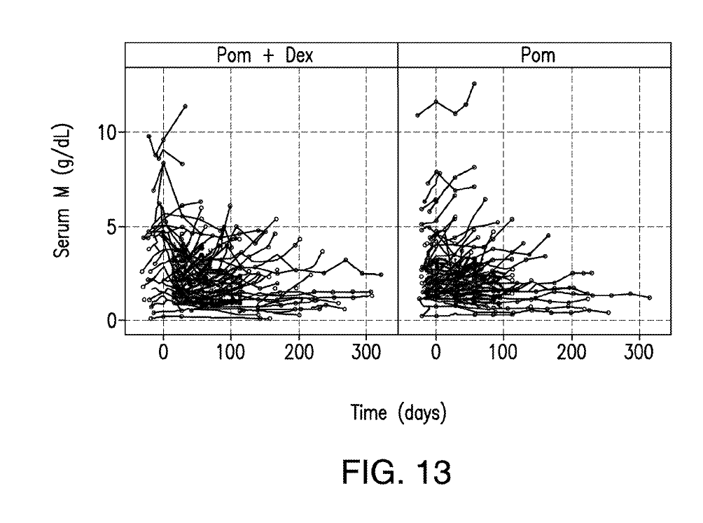

The simulations were performed based on interim data from the Phase II part of the pomalidomide study delivered on Nov. 17, 2010 (see 5.1). In the pomalidomide single agent arm, only serum M-protein data prior to dexamethasone administration were considered. Overall, data were available for 187 patients: 94 entered in the pomalidomide single agent arm and 93 entered in the pomalidomide plus dexamethasone arm out of the 217 patient in the dataset. To be evaluable, patients needed to have at least two M-protein measurements to be analyzed using the empirical M-protein model to predict end-of-cycle 2 (week 8) change from baseline in M-protein. M-protein longitudinal profiles are illustrated in FIG. 13.

5.6.3 M-Protein Reduction at Week 8

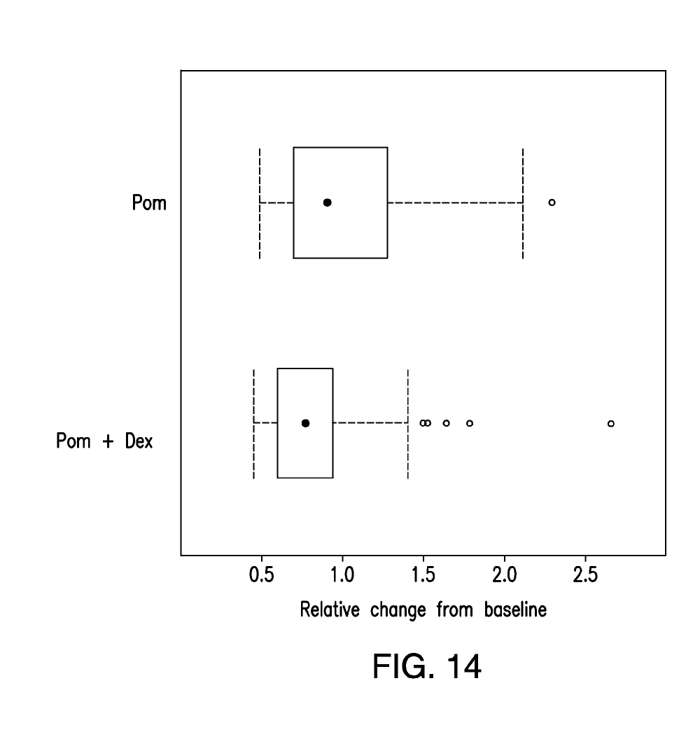

The empirical FDA tumor size model (Wang et al., Clin. Pharmacol. Ther. (2009), 86: 167-74) provided a good fit of M-protein data. Predicted serum M-protein relative change from baseline at end of cycle 2 (week 8) showed a better response in the pomalidomide plus dexamethasone combination arm compared to pomalidomide single agent (FIG. 14).

5.6.4 Clinical Endpoints: PFS and Survival

Simulations showed that that pomalidomide plus dexamethasone would perform better than pomalidomide single agent (Table IX).

TABLE IX
Expected PFS and survival in study MM-002

| | Median | | |
|---|---|---|---|
| | (week) | 2.5% | 97.5% |
| PFS | | | |
| Pom | 16.5 | 9.7 | 27.7 |
| Pom + Dex | 22.5 | 14.6 | 34.3 |
| Survival | | | |
| Pom | 67.8 | 45.8 | 101.3 |
| Pom + Dex | 78.3 | 53.5 | 116.1 |

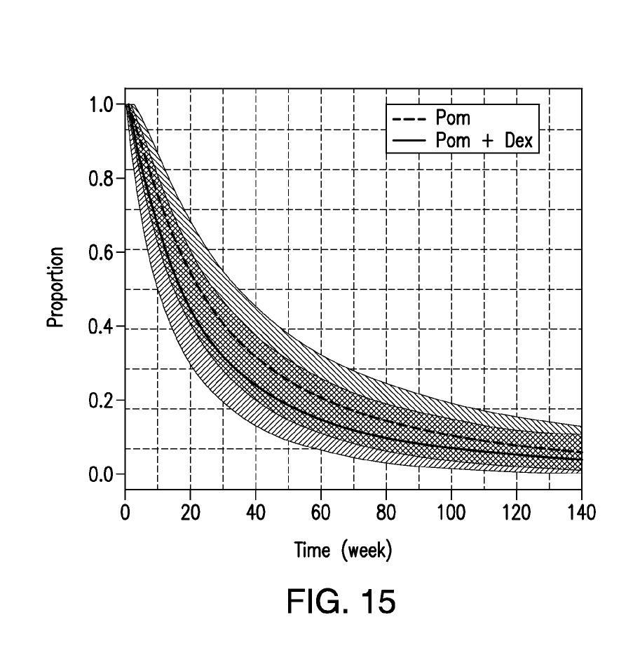

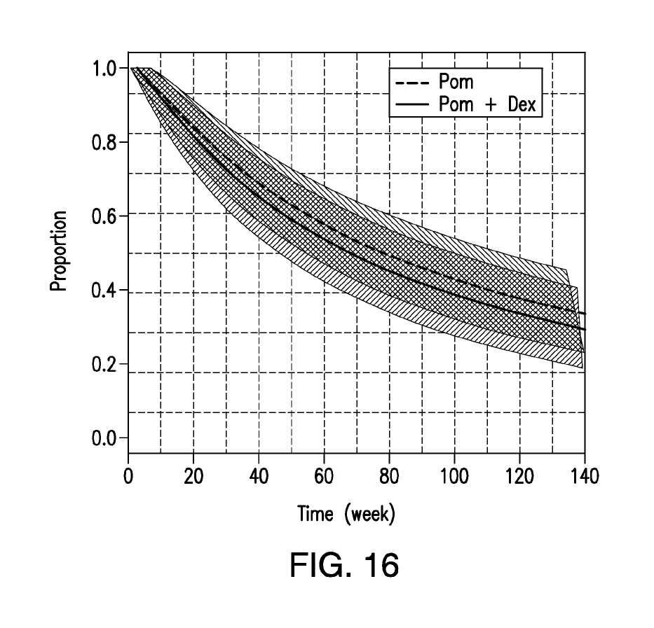

However there is overlap between the expected 95% confidence intervals as illustrated for PFS (FIG. 15) and survival (FIG. 16).

The simulations of survival and PFS were performed based on interim clinical data for the treatment of multiple myeloma with pomalidomide, specifically end of cycle 2 (week 8) M-protein change from baseline and patients characteristics using the survival drug independent model and the PFS “lenalidomide plus dexamethasone” model. Data in the pomalidomide arm were limited to data prior to dexamethasone in order to simulate expected clinical endpoints for pomalidomide single agent. Model simulations indicate that pomalidomide plus dexamethasone would perform better than pomalidomide single agent:

Median PFS: 22.5 (14.6-34.3) weeks vs. 16.5 (9.7-27.7) weeks

Median survival: 78.3 (53.5-116.1) weeks vs. 67.8 (45.8-101.3) weeks.

A modeling framework has been developed combining tumor growth inhibition and clinical endpoint models that can be used to support development decisions. Week 8 change in M-Protein (p<0.00001), ECOG performance status (p<0.0009), baseline albumin, hemoglobin and creatinine levels (p<0.01) were significant independent predictors of survival when week 8 change in M-Protein (p<0.00001) and baseline hemoglobin (p<0.001) were significant independent predictors of PFS. Modeling and simulation enables the use of the change in M-protein level as a continuous longitudinal biomarker for drug effect in multiple myeloma studies. The drug considered may be any therapeutic candidate for the treatment of multiple myeloma, including but not limited to immunomodulatory compounds as described herein. Data indicate encouraging results for 4-amino-2-(2,6-dioxopiperidin-3-yl)isoindole-1,3-dione (pomalidomide, Actimid®) in a refractory multiple myeloma patient population. Pomalidomide may also be administered in combination with dexamethasone.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method of predicting a patient response to a treatment of multiple myeloma, comprising:
obtaining tumor cells from the patient;
culturing the tumor cells in the presence or absence of an immunomodulatory compound;
measuring the level of a biomarker in the tumor cells; and
comparing the levels of the biomarker in the tumor cells cultured in the presence of an immunomodulatory compound to the levels of the biomarker in the tumor cells cultured in the absence of the immunomodulatory compound;
wherein a decreased level of the biomarker in the presence of the immunomodulatory compound indicates the likelihood of an effective patient response to the immunomodulatory compound.

2. A method of monitoring a patient response to a treatment of multiple myeloma, comprising:
obtaining a biological sample from the patient;
measuring the level of a biomarker in the biological sample;
administering an immunomodulatory compound to the patient;
thereafter obtaining a second biological sample from the patient;
measuring the level of the biomarker in the second biological sample; and
comparing the levels of the biomarker obtained from the first and second biological samples;
wherein a decreased level of the biomarker in the second biological sample indicates the likelihood of an effective patient response.

3. A method for monitoring a patient compliance with a drug treatment protocol for multiple myeloma, comprising:
obtaining a biological sample from said patient;
measuring the level of a biomarker in said sample; and
determining if the level of the biomarker is decreased in the patient sample compared to the level of the biomarker in an untreated control sample;
wherein a decreased level indicates patient compliance with said drug treatment protocol.

4. The method of claim 1, 2, or 3, wherein the biomarker is selected from the group consisting of M-protein, albumin, creatinine, hemoglobin, beta-2-microglobulin, and combinations thereof.

5. The method of claim 1, 2, or 3, wherein the biomarker is M-protein.

6. The method of claim 2 or 3, wherein the biological sample is blood or urine.

7. The method of claim 2 or 3, wherein the biological sample is blood.

8. The method of claim 5, wherein the immunomodulatory compound is 4-(amino)-2-(2,6-dioxo(3-piperidyl))-isoindoline-1,3-dione, or a pharmaceutically acceptable salt, solvate or stereoisomer thereof.

9. The method of claim 8, wherein the immunomodulatory compound is 4-(amino)-2-(2,6-dioxo(3-piperidyl))-isoindoline-1,3-dione.

10. The method of claim 9, wherein the treatment further comprises administering a therapeutically effective amount of a second active agent.

11. The method of claim 10, wherein the second active agent is dexamethasone.

12. The method of claim 9, wherein the compound is administered in an amount of from about 0.5 mg to about 4 mg per day.

13. The method of claim 12, wherein the compound is administered in an amount of about 2 mg per day.

14. The method of claim 9, wherein the compound is administered cyclically.

15. The method of claim 14, wherein one cycle comprises four weeks.

16. The method of claim 15, wherein one cycle comprises the administration of the compound for 21 days followed by seven days rest.

17. The method of claim 15, wherein the compound is administered in an amount of from about 0.5 mg to about 4 mg per day for 21 days followed by seven days rest in a 28 day cycle.

18. The method of claim 9, wherein the multiple myeloma is refractory myeloma, relapsed myeloma, or relapsed and refractory Dune-Salmon stage III multiple myeloma.

19. The method of claim 11, wherein dexamethasone is administered in an amount of about 40 mg once daily on days 1 to 4, 9 to 12, and 17 to 20 every 28 days.

20. The method of claim 11, wherein dexamethasone is administered in an amount of about 40 mg once daily on days 1, 8, 15 and 22 every 28 days.

21. The method of claim 11, wherein the compound is orally administered in an amount of from about 0.5 mg to about 2 mg per day on days 1 through 28 every 28 days, and dexamethasone is administered in an amount of about 40 mg once daily on days 1, 8, 15 and 22 every 28 days.

22. The method of claim 9, wherein the compound is administered orally.

23. The method of claim 22, wherein the compound is administered in the form of a capsule or tablet.
